(19) United States
(12) Patent Application Publication
(10) Pub. No.: US 2013/0101628 A1
(43) Pub. Date: Apr. 25, 2013

(54) NOVEL VEGF MIMETIC PEPTIDE-BASED SCAFFOLDS FOR THERAPEUTIC ANGIOGENESIS AND METHODS FOR THEIR USE

(71) Applicants: Matthew J. Webber, Cambridge, MA (US); Jörn Tongers, Hannover (DE); Douglas W. Losordo, Chicago, IL (US); Samuel I. Stupp, Chicago, IL (US)

(72) Inventors: Matthew J. Webber, Cambridge, MA (US); Jörn Tongers, Hannover (DE); Douglas W. Losordo, Chicago, IL (US); Samuel I. Stupp, Chicago, IL (US)

(73) Assignee: NORTHWESTERN UNIVERSITY, Evanston, IL (US)

(21) Appl. No.: 13/459,234

(22) Filed: Apr. 29, 2012

Related U.S. Application Data
(60) Provisional application No. 61/480,450, filed on Apr. 29, 2011.

(51) Int. Cl. C07K 14/00

(52) U.S. Cl. CPC C07K 14/001
USPC 424/400; 530/300; 530/326; 514/13.3

(57) ABSTRACT

Disclosed herein is a completely synthetic cell-free therapy based on peptide amphiphile nanostructures designed to mimic the activity of vascular endothelial growth factor (VEGF), one of the most potent angiogenic signaling proteins. The VEGF-mimetic filaments disclosed herein were found to induce phosphorylation of VEGF receptors and induce pro-angiogenic behavior in endothelial cells, indicated by an enhancement in proliferation, survival and migration in vitro.

### RELATED APPLICATIONS

This application is a non-provisional of U.S. Provisional Application No. 61/480,450, filed Apr. 29, 2011, which is incorporated herein by reference.

### STATEMENT OF GOVERNMENTAL SUPPORT

This invention was made with U.S. Government support under NH-I Grant Nos. 1RO1-EB003806-04, HL-53354, HL-57516, HL-77428, HL-63414, HL-80137, PO1HL-66957. The U.S. Government has certain rights in this invention.

### REFERENCE TO SEQUENCE LISTING

The official copy of the sequence listing is submitted herewith in both paper and electronic form. The latter contains an ASCII formatted sequence listing with a file named NAN_{—}007P_SEQ_LIST.txt, created on Apr. 28, 2011, and having a size of 48 KB submitted concurrently with the specification. The sequence listing contained in this file is part of the specification and is herein incorporated by reference in its entirety.

### FIELD OF THE INVENTION

The present invention relates generally to novel VEGF-mimetic peptide-based scaffolds and their use in the context of therapeutic angiogenesis. More particularly, the present invention relates to self-assembling peptide amphiphiles (PAs) that include a terminal bioactive domain that mimics VEGF and thereby binds to and/or activates one or more VEGF receptors so as to promote vascular and endothelial regeneration. Nanofiber scaffolds composed of such mimetic PAs (e.g., VEGF-PA) are demonstrated hereinto to activate primary VEGF receptors, induce pro-angiogenic effects in vitro, trigger angiogenic responses in vivo, and demonstrate efficacy in in vivo models of ischemia. Accordingly, the peptide nanostructures of the present invention are expected to have utility in connection with ischemic tissue repair, wound healing, and the treatment of cardiovascular disease, including coronary and peripheral artery disease and may further be incorporated with traditional tissue engineering strategies to enhance host-derived vascularization and endothelialization of implanted tissue, biomaterial constructs, or medical devices such as stents and prostheses.

### BACKGROUND OF THE INVENTION

Ischemic tissue disease remains one of the foremost causes of morbidity and mortality worldwide [1]. Established clinical practice for treatment of occluded arteries is rapid circulatory restoration to limit progressive organ dysfunction. However, revascularization is not consistently successful, leaving a tremendous medical need for new therapeutic approaches that regenerate ischemic tissue. One emerging approach is to enhance microvasculature perfusion of ischemic tissue through the delivery of angiogenic factors, termed therapeutic angiogenesis. The mechanisms involved in angiogenesis have been extensively studied in recent years, and its regulation has been shown to involve complex cascades of many signaling molecules and growth factors [2]. Vascular endothelial growth factor (VEGF) is among the most potent, yet rate-limiting, of the angiogenic factors [3]. Thus, a number of pre-clinical studies and clinical trials have focused on VEGF protein- or gene-based therapies to treat ischemic diseases [4-6]. Protein-based therapies, while demonstrating success in several pre-clinical trials, are limited by a lack of tissue-specific targeting and inadequate temporal levels of the protein in the target zone [4, 7, 8]. Moreover, the costs associated with protein preparation and purification make these therapies, if feasible, extremely expensive at the clinical level [4]. Another delivery method for these potent proteins is gene therapy for cells in or at the periphery of the ischemic area, which then become the source of protein production. While these vector-based approaches are not necessarily as expensive as recombinant protein, limitations range from inadequate transduction efficiency with some vectors to safety issues with others [9]. Moreover, clinical trials using gene therapy to deliver angiogenic factors are yet to demonstrate convincing efficacy for treatment of cardiovascular diseases [4].

In the past years, a number of synthetic strategies have emerged as a means of modulating angiogenesis through small molecules, peptides, or antibodies. Many of these systems are designed to either antagonize native angiogenic signaling processes, for example in cancer therapies, or to promote angiogenic signaling as receptor agonists, in the case of treatments for ischemic diseases [10, 11]. Currently, only one synthetic oligopeptide has demonstrated the capability to mimic VEGF through the activation of its receptors [12, 13]. This peptide was designed based on the native □-helical receptor-binding domain of VEGF and was shown to signal in a manner consistent with native VEGF and compete with VEGF for binding to its receptors. Presentation of this peptide epitope on a biomaterial increased pro-angiogenic activity of encapsulated endothelial cells by stimulating proliferation and tubulogenesis in vitro[14]. While the synthetic oligopeptide addresses the major issue of protein production cost, it does not significantly improve on protein therapies in terms of tissue retention and sustained delivery. Thus, there remains a need for improved synthetic therapeutic strategies.

The field of bio-nanotechnolgy has emerged in recent years as a means to translate advancements in nanoscience into practical strategies for regenerative medicine [15, 16]. Supramolecular self-assembly of rationally designed molecules is one such strategy, highlighted by the work of Stupp et. al. and their development of a platform of self-assembling filamentous nanofibers formed from customizable peptide amphiphiles (PAs) that can be designed for specific biological targets [15-18]. The PAs of this platform are primarily composed of a hydrophobic alkyl segment covalently linked to a peptide sequence that contains at least two domains: an amino acid sequence that drives self-assembly of the molecules into nanofibers by promoting the formation of □-sheets [19, 20] and a customizable bioactive domain (normally a terminal domain) designed to interact with specific proteins, receptors, biopolymers or other cellular targets. Additional domains may incorporate, by design, charged amino acids to partially inhibit self-assembly until electrolytes in physiologic environments screen the charged residues and trigger PA self-assembly into high aspect ratio nanofibers. Upon screening, hydrophobic collapse drives the alkyl tails into the core of these nanofibers, resulting in the presentation of the bioactive domains on the fiber surface. The dimensions of these assembled PA nanofibers are on the order of 10 nanometers in diameter and their length on the order of microns, mimicking the size scale and architecture of filamentous structures in natural extracellular matrices. The high aspect ratio nanofibers can form gel networks at relatively low concentrations in aqueous media (1% by weight or even lower), allowing for three-dimensional entrapment of cells pre-suspended in aqueous PA solutions [21]. The filamentous PM of Stupp et al. have revealed great capacity to be bioactive in several animal disease models, including spinal cord injury and cartilage damage [22, 23]. The potential for in vivo efficacy of PAs over a broad scope of regenerative targets is probably linked to their unique supramolecular architecture and molecular dynamics. Their high aspect ratio and high surface area with displayed signals at controlled density are two important characteristics that facilitate enhanced biological signaling [24, 25]. These nanostructures also integrate high degrees of order for effective signal display, but their extensive internal hydration also offers the necessary dynamics to promote successful binding with receptors and ligands [26]. Furthermore, these PA-based therapies can be delivered non-invasively as easily injectable liquids that become solid nanostructures in situ. They have also demonstrated biocompatibility, and desirable rates of degradation and tissue clearance [23, 27].

Given the drawbacks of protein- and gene-based therapies, the present inventors sought to engineer pro-angiogenic peptide amphiphiles capable of mimicking angiogenic growth factors to be developed as therapies for cardiovascular diseases. As VEGF is one of the most frequently employed proteins for therapeutic angiogenesis of ischemic tissue, the present invention focused on the development of a VEGF-mimetic PA that could self-assemble into nanofibers that effectively signal as VEGF does. The translation of the novel VEGF-PA of the present invention is explored herein as a potential therapy for regeneration of tissue in a model for critical ischemia.

### SUMMARY OF THE INVENTION

Accordingly, it is an object of the present invention to incorporate a VEGF-mimetic peptide sequence into the framework of a peptide amphiphile to yield a self-assembling structure having the requisite binding activity to activate VEGF receptors. As discussed in greater detail below, evaluation of the bioactivity in vitro and in vivo found the VEGF-PA structure of the present invention was observed to have enhanced bioactivity as compared to peptide alone. As a treatment for hind-limb ischemia, the VEGF-mimetic nanostructure of the present invention was found to enhance limb recovery following critical hind limb ischemia as assessed by tissue salvage and motor function of the ischemic hind-limb. These findings are associated with an improved blood flow measure by laser Doppler imaging. Immunohistological evaluation revealed a significantly enhanced microcirculation. Given that the major drawbacks of protein-based therapies lie in the excessive production costs and poor target tissue retention, the use of peptide amphiphiles to present bioactive VEGF-mimetic epitopes constitutes a significant improvement over the prior art, offering the benefits of large-scale synthesis, inexpensive production, enhanced tissue retention, increased bioavailability of the signal, and capacity to localize the signal within the nanofiber. The self-assembling VEGF-PA of the present invention is also able to efficiently signal cells in a manner consistent with VEGF signaling through a network of high aspect-ratio entangled fibers that can provide a more lasting signal in the host tissue due to prolonged residence time and high density of displayed epitopes. For example, whereas a single injection of VEGF has a short retention time within the tissue, the VEGF epitope-presenting PAs of the present invention remain in the tissue and bioavailable on the order of weeks. Further, this totally synthetic approach allows for these molecules to be produced through a highly controlled process on the scale of multiple grams for a cost much less than that associated with the preparation and purification of recombinant factors.

In view of the above, it is an objective of the present invention is to provide a peptide amphiphile composed of the following segments: (1) a mimetic peptide segment comprising an agonist that binds to or interacts with a pro-angiogenic growth factor receptor; (2) a spacer segment that confers both solubility and flexibility to the peptide; (3) a beta-sheet forming, structural peptide segment, and (4) a non-peptide lipophilic segment. In the context of the present invention, a VEGF agonist that targets a VEGF receptor is particularly preferred. Accordingly, the VEGF mimetic peptides KLTWQELYQLKYKGI-NH2 (SEQ ID NO: 2) and d-(IGKYKLQYLEQWTLK) (SEQ ID NO: 28) are particularly preferred.

Preferred spacer segments have the form (Xaa)m-(Gly)n, wherein m and n are integers that independently range between 0 and 5 and Xaa is an amino acid with basic side-chains. A particularly preferred spacer peptide is KKKG (SEQ ID NO: 6).

Preferred structural peptide segments have the form (XA)Na(XB)Nb wherein XA and XB are selected from A, L, V and G and Na and Nb are 2, 3 or 4. A particularly preferred structural peptide segment is VVAA (SEQ ID NO: 19).

Preferred lipophilic segments are single, saturated, linear acyl groups of the formula: CH3(CH2)n-2C(O)—, wherein n ranges from 6 to 22. A particularly preferred lipophilic segment is a saturated, palmitoyl acyl chain (i.e., CH3(CH2)14C(O), wherein n is 16). The lipophilic segment may optionally be covalently linked to the epsilon amine of a lysine residue.

Thus, it is objective of the present invention to provide a self-assembling peptide amphiphile having of the following structure:

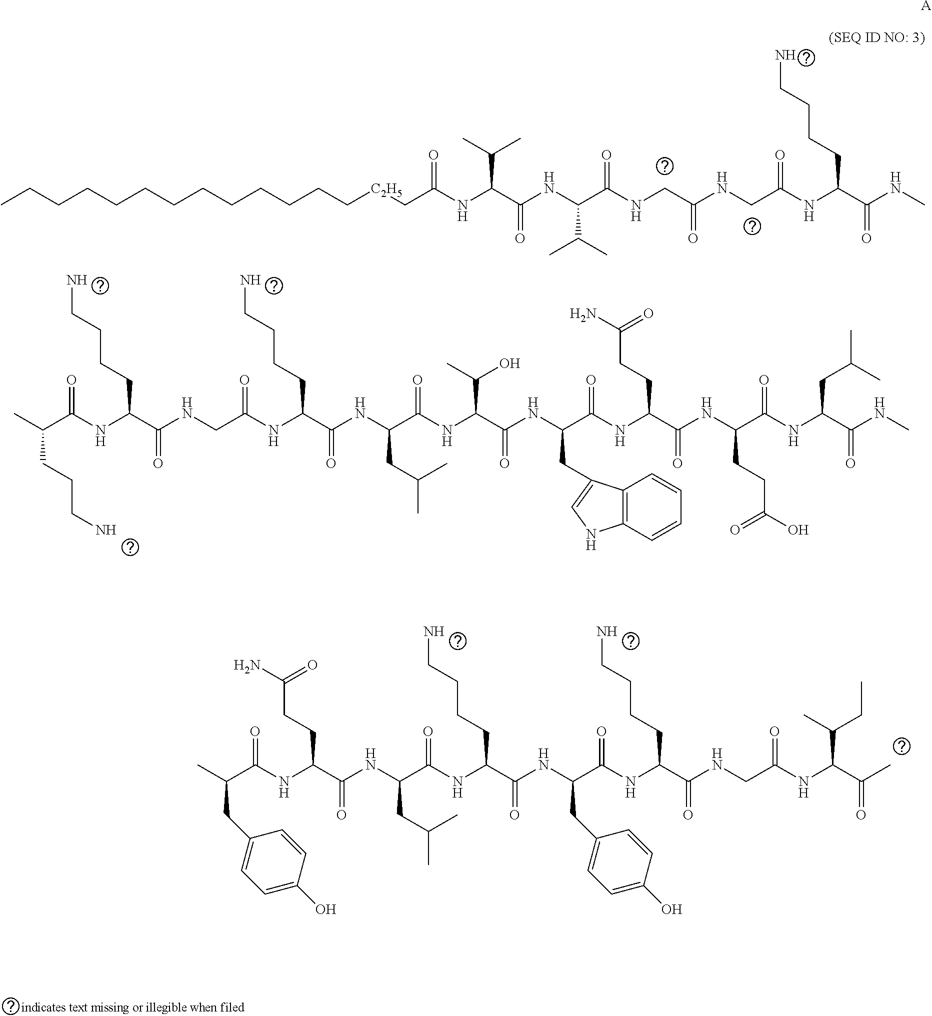

It is yet another objective of the present invention to provide a composition comprising one or more of a peptide amphiphile as described above, preferably dispersed in an aqueous medium.

It is yet a further objective of the present invention to provide a method of treating a patient afflicted with a disease or disorder characterized by poor circulation and blood vessel formation, the method including the step of administering a peptide amphiphile or composition as described above to a target site within the body of the patient. In one preferred embodiment, the disease is peripheral artery disease and the target site is an occluded vessel. In an alternate preferred embodiment, the disorder is ischemia and the target site is a chronic ischemic wound.

It is yet another objective of the present invention to provide a method of treating an ischemic disease or defect in a patient in need thereof, the method including the step of administering an afore-mentioned composition to the patient. In a preferred embodiment, the composition is administered intravenously or intramuscularly and the patient is a human or an animal such as a horse, dog, sheep, goat, or cow.

It is yet another objective of the present invention to provide a method of regenerating vasculature that includes the step of administering to a patient in need thereof one or more of the afore-mentioned compositions.

Finally, it is yet another objective of the present invention to provide a substrate coated with self-assembled micelles formed by a peptide amphiphile described above.

These and other objectives and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art having knowledge of various amphiphilic compounds, self-assembly techniques and peptide synthesis. Such objects, features, benefits and advantages will be apparent from the above as taken into conjunction with the accompanying examples, data, figures and all reasonable inferences to be drawn there from, alone or with consideration of the references incorporated herein.

### BRIEF DESCRIPTION OF THE DRAWINGS

Various aspects and applications of the present invention will become apparent to the skilled artisan upon consideration of the brief description of the figures and the detailed description of the present invention and its preferred embodiments that follows:

FIG. 1: Part (A) depicts the chemical structure of a preferred peptide amphiphile (PA) referred to herein as “VEGF-PA” (SEQ ID NO:3), designed to assemble into cylindrical nanostructures (G). The VEGF-PA form nanofibers, visualized by Cryogenic Transmission Electron Microscopy (Cryo-TEM) (B) and entangled nanofiber gel networks imaged by Scanning Electron Microscopy (SEM) (C). Circular Dichroism (CD) of the VEGF-PA demonstrating a-helical secondary structure (D) and melting analysis performed about the 220 nm a-helical signature for VEGF-PA (E) and the peptide epitope control (F) are also presented. The results demonstrate that the VEGF-PA (E) stabilizes the active a-helical conformation of the bioactive peptide as compared to the peptide alone (F), thereby confirming the enhanced stability of the bioactive conformation of the epitope when incorporated into the PA.

FIG. 2: Part (A) depicts the results from an ELISA assessing the level of specific phosphorylation to VEGF receptor-1 (VEGFR1) induced by the VEGF-PA (SEQ ID NO: 3), which demonstrates a significant increase for treatment with VEGF-PA compared to all other groups evaluated. Part (B) depicts the results from an ELISA assessing the level of specific phosphorylation to VEGF receptor-2 (VEGFR2) induced by the VEGF-PA, which demonstrates a significant increase for treatment with VEGF-PA compared to a mutant PA control and an untreated control. Part (C) depicts the time-dependent results from an ELISA measuring phosphorylation in response to stimulation with VEGF-PA, showing an early rise in phosphorylation followed by a decrease over time.

FIG. 3: Part (A) depicts the effect of stimulation with VEGF-PA (SEQ ID NO: 3) as compared to VEGF alone, a control PA, and an untreated control on the proliferation of human endothelial cells in vitro. VEGF-PA compared to no increase for the peptide alone, control PA, and untreated control. A significant increase in proliferation was observed in cells stimulated with VEGF-PA as compared to no increase for the peptide alone, control PA, and untreated control. Part (B) depicts the effect of stimulation with VEGF-PA as compared to VEGF alone, a control PA, and an untreated control on the survival of human endothelial cells after serum starvation in vitro. A significant increase in survival (less cell death) was observed in cells stimulated with VEGF-PA as compared to the peptide alone, control PA, and untreated control. Part (C) depicts the effect of stimulation with VEGF-PA as compared to VEGF alone, a control PA, and an untreated control on the migration of human endothelial cells into a scratch wound in vitro. A significant increase in migration (faster wound closure) was observed in cells stimulated with VEGF-PA as compared to the control PA and untreated control with representative images for cells treated with (D) VEGF-PA (SEQ ID NO: 3), (E) VEGF peptide (SEQ ID NO: 4), (F) mutant PA (SEQ ID NO: 5), (G) no treatment.

FIG. 4 presents the results from an in vivo chicken chorioallantoic membrane (CAM) assay for angiogenesis. Materials were coated onto coverslips and placed onto the CAM. Part (A) shows the quantification of the number of blood vessels intersecting the perimeter of the glass coverslip expressed relative to the number at the time of coverslip application to reveal a significant increase in the number of vessels when the CAM is stimulated with VEGF-PA (SEQ ID NO: 3) for each day of follow-up thereafter. Parts (B-E) provide representative images of the treated CAM for application of VEGF-PA (SEQ ID NO: 3)(B), VEGF peptide (SEQ ID NO: 4)(C), mutant PA (SEQ ID NO: 5)(D), and saline (E). Coverslips are outlined for visualization and an arrow indicates the formation of a typical angiogenic response in this assay characterized by vascular reorganization, leakage, and vessel spoke patterns.

FIG. 5 presents the results from an in vivo hind-limb study examining the tissue salvage score (A) and motor function score (B) of the various treatment groups over time, as well as the endpoint analysis at day 28 of failure time for a Rota Rod motor functional performance test (C). Laser Doppler perfusion imaging (D) for mouse hind-limb ischemia studies quantified as the perfusion ratio of treated to untreated limb along with representative LDPI images form the same animal at day 0 and day 28 for treatments with VEGF-PA (SEQ ID NO: 3), VEGF peptide alone (SEQ ID NO: 4), a mutant PA (SEQ ID NO: 5), and an untreated control. Significant is shown for the VEGF-PA relative to the other treatments (A, B, and D) and for other treatments compared to the VEGF-PA treatment (C).

FIG. 6: Part (A) depicts the density of capillaries in muscle of the ischemic limb obtained from immunohistochemical staining for CD31+vessels. The results reveal a significant increase in blood vessels within the ischemic tissue for animals treated with VEGF-PA (SEQ ID NO: 3) as compared to treatment with the peptide alone, a mutant PA, or no treatment. Representative histological images from animals treated with (B) VEGF-PA (SEQ ID NO: 3), (C) VEGF Peptide (SEQ ID NO: 4), (D) mutant PA (SEQ ID NO: 5), and (E) untreated animals stained specifically for endothelial cells (CD3 1 antibody, green), vascular smooth muscle cells (smooth muscle actin, red), and nuclei (blue).

### DETAILED DESCRIPTION OF THE PREFERRED EMBODIMENTS

Although any methods and materials similar or equivalent to those described herein can be used in the practice or testing of embodiments of the present invention, the preferred methods, devices, and materials are now described. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the present invention which will be limited only by the appended claims.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the present invention, the following definitions apply:

As used herein and in the appended claims, the singular forms “a”, “an” and “the” include plural reference unless the context clearly dictates otherwise. Thus, for example, reference to a “cell” is a reference to one or more cells and equivalents thereof known to those skilled in the art, and so forth.

As used herein, the term “nanofiber” refers to an elongated or threadlike filament having a diameter of equal to or less than 100 nanometers.

As used herein, the term “cylindrical micelle” refers to a colloidal aggregate with a non-spherical, high-aspect-ratio shape (length/diameter >10), composed of amphiphilic molecules in which the hydrophobic (or lipophilic) part of the amphiphiles forming the micelle tends to locate away from the polar phase (e.g. water) while the polar parts of the molecule (head groups) tend to locate at the micelle-solvent interface.

As used herein, the term “physiological conditions” refers to the range of conditions of temperature, pH and tonicity (or osmolality) normally encountered within tissues in the body of a living human.

As used herein, the terms “self-assemble” and “self-assembly” refer to formation of a discrete, non-random, aggregate structure from component parts; said assembly occurring spontaneously through random movements of the components (e.g. molecules) due only to the inherent chemical or structural properties of those components.

As used herein, the terms “scaffold” and “matrix” refer interchangeably to a natural or synthetic structure or meshwork of structures with open porosity that is extended in space and provides mechanical or other support for the growth of living tissue, either in the body or in vitro.

As used herein, the term “gel” refers to a semi-solid, viscoelastic material (capable of resisting some mechanical stress without deformation), which is formed by the coagulation of a colloidal liquid, composed of a fibrous matrix and fluid-filled interstices.

As used herein, the term “peptide amphiphile” refers to a molecule that, at a minimum, includes a non-peptide lipophilic segment, and a peptide segment having at least six amino acid residues. The peptide amphiphile may express a net charge at physiological pH, either a net positive or negative net charge, or may be zwitterionic (i.e., carrying both positive and negative charges).

A. Mimetic Peptide Amphiphiles

The mimetic receptor-binding peptide amphiphiles of the present invention include, at a minimum, the following segments: (1) a mimetic peptide segment that binds to or interacts with a receptor for a pro-angiogenic growth factor such as VEGF; (2) a spacer segment that confers both solubility and flexibility to the peptide; (3) a structural peptide segment that confers the molecule with the ability to form a beta-sheet secondary structure, and (4) a lipophilic segment, composed generally of a single alkyl chain.

As used herein and in the appended claims, the term “lipophilic segment” refers to the hydrocarbon moiety disposed on or about the terminus of the peptide amphiphile. This lipophilic segment may be herein and elsewhere referred to as the hydrophobic component or hydrophobic segment. The lipophilic segment should be of a sufficient length to provide amphiphilic behavior and micelle formation in water or another polar solvent system.

Accordingly, in the context of the present invention, the lipophilic segment is preferably composed of a single, saturated, linear acyl chain of the formula: CH3(CH2)n-2C(O)—, where n=6−22. In a preferred embodiment, this lipophilic segment may be covalently linked via acylation of a fatty acid to N-terminal amine of the peptide, or to the epsilon amine of a C-terminal lysine residue. A particularly preferred lipophillic segment consists of a saturated, palmitoyl acyl chain (i.e., CH3(CH2)14C(O)— where n=16).

As used herein and in the appended claims, the term “spacer segment” refers to an intermediate amino acid sequence of the peptide amphiphile molecule that confers both solubility and flexibility to the peptide. In a preferred embodiment, the spacer segment includes the amino acid sequence (Xaa)m-(Gly)n wherein m and n are integers that range between 0 and 5, more preferably between 1 and 3, wherein Xaa is an amino acid residue selected from those with basic side-chains, including, for example, lysine (K), histadine (H) and arginine (R). In the context of the present invention, one particularly preferred spacer segment has the amino acid sequence KKKG (SEQ ID NO: 6). This spacer segment is utilized in the exemplary peptide amphiphile of SEQ ID NO: 3, which has the following structure:

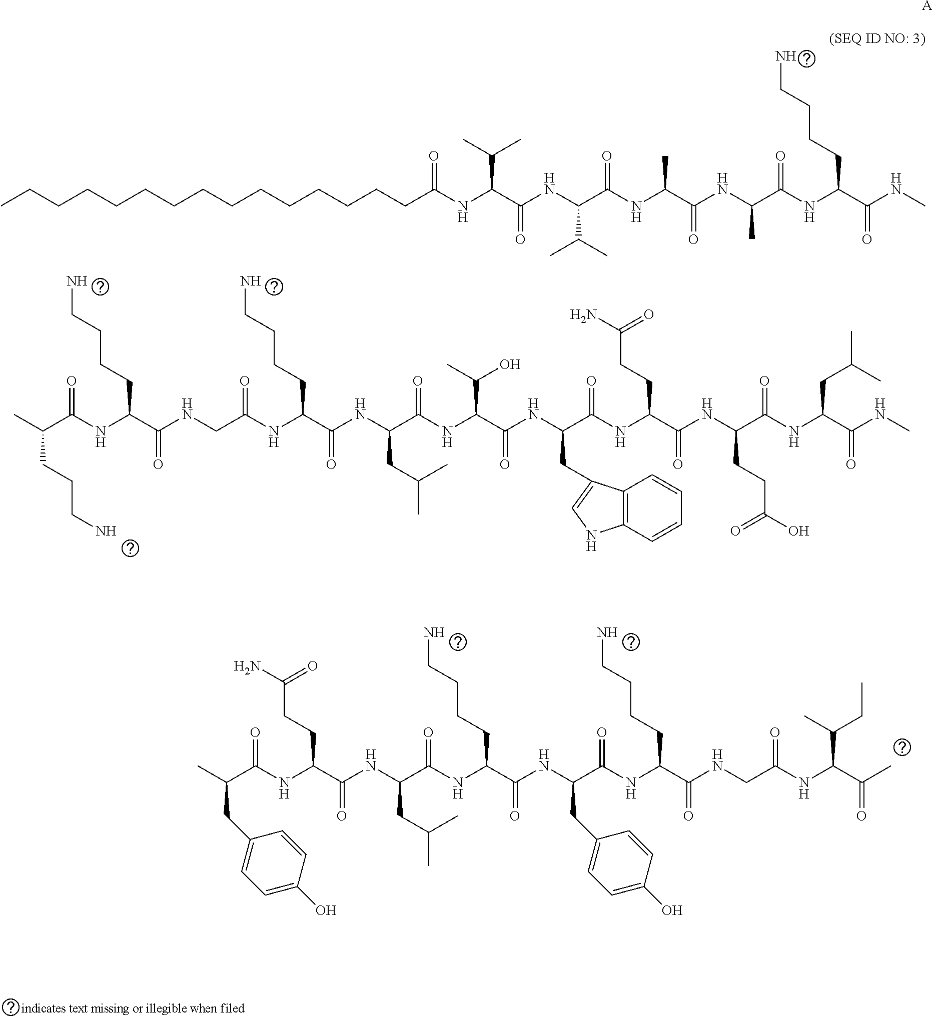

Increasing or decreasing the length of the basic amino acid residue's side-chain can also modify the solubility of peptide amphiphiles containing that residue, as can changing the number of amine groups on the side-chain. Accordingly, the present invention contemplates a peptide-amphiphile molecule wherein Xaa (as described above) is an alpha-substituted amino acid with 1 to 5, more preferably 3 to 4 carbon atoms between the alpha carbon and one or more amine residues. In a preferred embodiment, Xaa is selected from ornithine or lysine.

As used herein and in the appended claims, the term “structural peptide segment” refers to the intermediate amino acid sequence of the peptide amphiphile molecule generally composed of three to ten amino acid residues with non-polar, uncharged side chains, selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), Gly (G), (listed in order of their propensity to form beta sheets). However, non-naturally occurring amino acids of similar beta-sheet forming propensity may also be used. In a more preferred embodiment, a strong and a weak beta sheet former are used in combination, for example taking the form (XA)Na(XB)Nb, where XA and XB are selected from A, L, V and G, Na and Nb are 2, 3 or 4. Illustrative examples include (SEQ ID NOs: 7-26)

| | | | |
|---|---|---|---|
| VVVAAA | AAAVVV | LLLAAA | VVVVVV |
| VVVLLL | LLLVVV | AAAAAA | AAAAGGG |
| LLLLLL | AAAGGG | LLLGGG | AAALLL |
| VVAA | VVVAA | VVAAA | VVVVAA |
| AAVV | AAVVV | AAAVV | AAVVVV |

In the context of the present invention, one particularly preferred structural peptide segment has the amino acid sequence AAVV (SEQ ID NO: 23), which is utilized in the exemplary peptide amphiphile of SEQ ID NO: 3, which has the following structure:

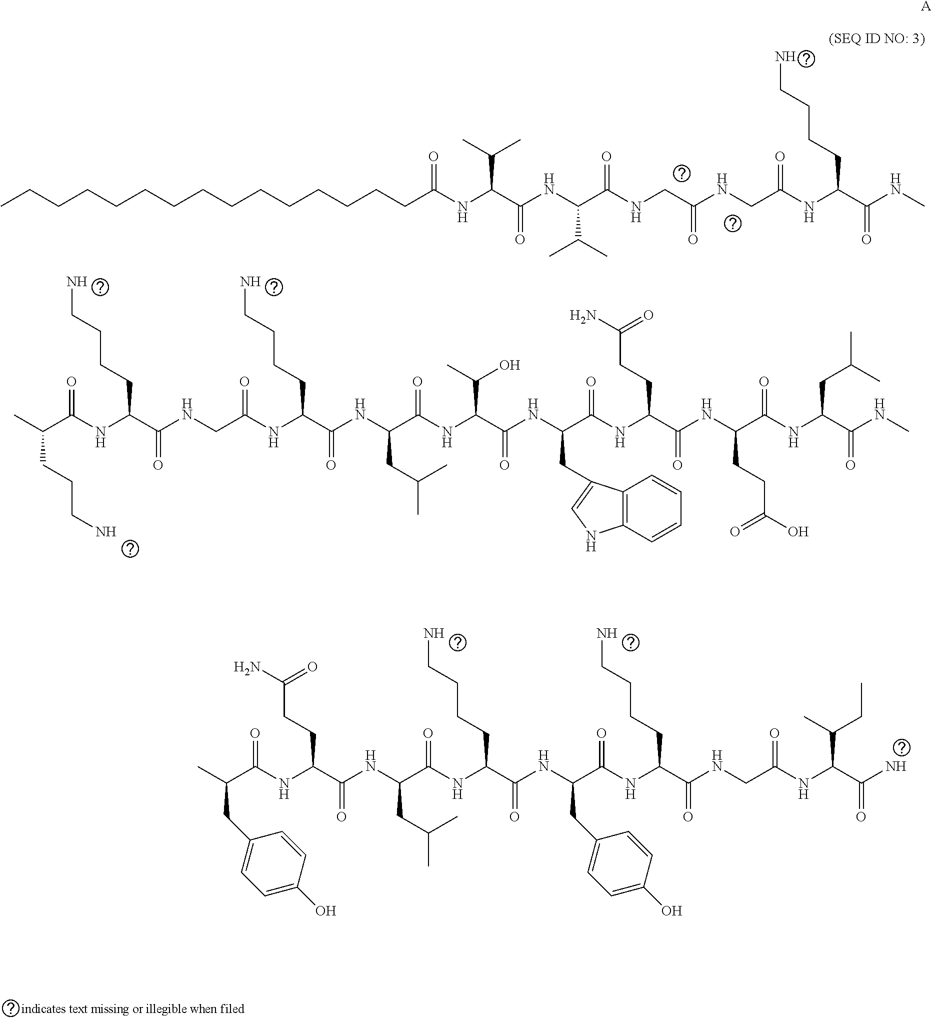

As used herein, the term “growth factor” refers to the broad class of bioactive polypeptides that control and regulate a variety of endogenous biological and cellular processes, such as cell-cycle progression, cell differentiation, reproductive function, development, motility, adhesion, neuronal growth, bone morphogenesis, wound healing, immune surveillance and cell apoptosis. Growth factors typically operate by binding to specific receptor sites on the surface of target cells. Growth factors include, but are not limited to, cytokines, chemokines, polypeptide hormones and the receptor-binding antagonists thereof. Examples of well known growth factors include but are not limited to:
Transforming growth factor beta (TGF-β);
Bone Morphogenic Protein (BMP);
Interleukin-17;
Transforming growth factor alpha (TGF-α);
Cartilage oligomeric matrix protein (COMP);
Cell Density Signaling Factor (CDS);
Connective tissue growth factor (CTGF);
Epidermal growth factor (EGF);
Erythropoietin (EPO);
Fibroblast growth factor (FGF);
Glial Derived Neurotrophic Factors (GDNF);
Granulocyte-colony stimulating factor (G-CSF);
Granulocyte-macrophage colony stimulating factor (GM-CSF);
Growth differentiation factor (GDF);
Myostatin (GDF-8);
Hepatocyte growth factor (HGF];
Insulin-like growth factor (IGF);
Macrophage inhibitory cytokine-1 (MIC-1);
Placenta growth factor (PIGF);
Platelet-derived growth factor (PDGF);
Thrombocyte concentrate (PRP);
Thrombopoietin (TPO);
Vascular endothelial growth factor (VEGF);
Activin and Inhibin;
Coagulogen;
Follitropin;
Gonadotropin and Lutropin;
Mullerian Inhibiting Substance (MIS) also called: Anti-Müllerian hormone (AMH) Müllerian inhibiting factor (MIF) and Mullerian inhibiting hormone (MIH);
Nodal and Lefty; and
Noggin

Therapeutic molecules that regulate, induce or participate in useful biological processes in the body, including those listed above, are often categorized or classified according to their particular structure or function. For example, immunoregulatory proteins secreted by cells of the immune system, such as interleukin and interferon, are often referred to as cytokines. Other categories of regulatory molecules include, but are not limited to:
morphogens (e.g., molecules that regulate or control the formation and differentiation of tissues and organs);
chemokines (e.g., any of a group of cytokines produced by various cells, as at sites of inflammation, that stimulate chemotaxis in white blood cells such as neutrophils and T cells);
hormones (e.g., a product of living cells that circulates in body fluids such as blood and produces a specific, often stimulatory effect on the activity of cells, usually remote from its point of origin);
receptors (e.g., a molecule present on a cell surface or in the cell interior that has an affinity for a specific chemical entity, including both endogenous substances such as hormones and ligands as well as foreign materials, such as viral particles, that serves as an intermediary between the stimulating agent and the downstream physiological or pharmacological response thereto);
receptor-binding agonists (e.g., a chemical substance capable of combining with a specific receptor on a cell and initiating the same reaction or activity typically produced by the endogenous binding substance (such as a hormone)); and
receptor-binding antagonists (e.g., a chemical substance that reduces the physiological activity of another chemical substance (such as a hormone) by combining with and blocking one or more receptors associated therewith).
   While the present invention finds particular use in connection with angiogenic growth factors such as vascular endothelial growth factor (VEGF), fibroblast growth factor (FGF-2), and other pro-angiogenic cytokines, chemokines, and growth factors, those skilled in the art that the principles of the present invention may be readily applied to the activation of other growth factors.

In the context of the present invention, VEGF is particularly preferred. The term “VEGF” refers to a sub-family of growth factors, more particularly, the platelet-derived growth factor family of cystine-knot growth factors. The VEGF family includes important signaling proteins involved in both vasculogenesis (the de novo formation of the embryonic circulatory system) and angiogenesis (the growth of blood vessels from pre-existing vasculature). VEGF family members include VEGF-A (associated with generic angiogenesis), VEGF-B (associated with embryonic angiogenesis), VEGF-C (associated with lymphangiogenesis), VEGF-D (required for the development of lymphatic vasculature surrounding lung bronchioles), and PIGF (important for vasculogenesis, associated with angiogenesis during ischemia, inflammation, wound healing, and cancer).

All members of the VEGF family stimulate cellular responses by binding to tyrosine kinase receptors (the VEGFRs) on the cell surface, causing them to dimerize and become activated through transphosphorylation, although to different sites, times and extents. All VEGF receptors have an extracellular portion consisting of 7 immunoglobulin-like domains, a single transmembrane spanning region, and an intracellular portion containing a split tyrosine-kinase domain. VEGF-A binds to VEGFR-1 (Flt-1) and VEGFR-2 (KDR/Flk-1). VEGF-C and VEGF-D are ligands for a third receptor (VEGFR-3), which mediates lymphangiogenesis.

It is a goal of the present invention to provide pro-angiogenic peptide amphiphile-based nanofibers that mimic the activity of angiogenic growth factors, particularly those of the VEGF family. Accordingly, as used herein and in the appended claims, the terms “mimetic segment” and “mimetic peptide” refers to a C-terminally disposed peptide sequence that mimics the receptor binding epitope of an angiogenic growth factor, particularly a VEGF epitope, and thus interacts with one or more angiogenic growth factor receptors, particularly VEGFR-I (Flt-1), VEGFR-2 (KDR/Flk-1), and VEGFR-3, to activate the signaling pathway. In this manner, the mimetic segment is analogous to a peptide “agonist”, a moiety that binds to a cell receptor and triggers a response by that cell. Upon self-assembly, the mimetic peptide is exposed at the surface of the nanofiber, thereby serving as a bioactive signal presented to the environment.

A particularly preferred mimetic peptide suitable for use in the context of the peptide amphiphiles of present invention is KLTWQELYQLKYKGI (SEQ ID NO: 2).

Reversing the polarity of the above sequence along with optionally inverting the stereochemistry of one or more amino acids may alter the pharmacological activity of the PA in a beneficial manner, for example by reversing the twist of the alpha helical structure or enhancing VEGF receptor signalling activity while resisting or reducing proteolytic degradation. Thus the mimetic peptides IGKYKLQYLEQWTLK (SEQ ID NO: 27) and D(IGKYKLQYLEQWTLK) (SEQ ID NO: 28)are preferred embodiments of the present invention.

As will be understood by one skilled in the art of peptide chemistry, other variations on the mimetic peptide sequence are possible and may lead to peptide amphiphiles with increased or decreased strength and selectivity of binding to the growth factor receptors, particularly VEGF receptors. These include substituting one or more of the non-polar amino acid residues (leucine (L), isoleucine (I),), with another, similarly non-polar residue, such as valine (V) or alanine (A); exchanging glutamine (Q) for asparagine (N); exchanging tyrosine (Y) with tryptophan (W) or phenylalanine (F); or substituting a positively charged lysine (K) residue with an arginine (R) residue (or vice versa).

In addition, cyclic peptides, formed through cross-linking of two or more amino acid residues in the above described peptides, may be useful for the applications described above. While not intending to be bound by theory, such cross-linking results in cyclic presentation of the receptor binding domain (epitope), which may in turn protect the C-terminal residue from enzymatic degradation, and thus result in enhanced biological signalling or receptor binding by the peptide.

Peptide sequences of the present invention include amino acid residues that may be subject to post-synthesis modification. For example, the de-amidation of glutamine (Q), via a succinimide intermediate is a common post-translational protein modification resulting in the transformation of the Gln side-chain to that of glutamic acid, Asp (E) This modification is associated in some instances with an altered (enhanced or reduced) effect on biological activity of the substrate protein. Moreover, synthetic peptides containing Gln residues can undergo de-amidation during manufacturing, particularly when exposed to alkaline pH and elevated temperatures. In the case of therapeutic peptides, this process may lead to altered (enhanced or reduced) efficacy. Thus, in one embodiment, the above VEGF mimicking sequences may be modified such that one or more Q residues are replaced with E, for example, KLTWEELYELKYKGI (SEQ ID NO: 29). In a preferred embodiment, these modified sequences are incorporated into a peptide amphiphile as described previously, such as VVAA-KKKG-KLTWEELYELKYKGI (SEQ ID NO: 30).

Amino acids useful in the peptide amphiphiles of the present invention include, but are not limited to, naturally occurring amino acids and artificial amino acids. Incorporation of artificial amino acids such as beta or gamma amino acids and those containing non-natural side chains, and/or other similar monomers such as hydroxyacids are also contemplated, with the effect that the corresponding component is peptide-like in this respect.

The peptide amphiphile molecules and compositions of the present invention can be synthesized using preparatory techniques well-known to those skilled in the art, preferably, by standard solid-phase peptide synthesis, with the addition of a acyl derivative of a fatty acid in place of a standard amino acid at the N-terminus of the peptide, or at the epsilon amine of a lysine side-chain, utilizing orthogonal protecting groups, as would be known to one skilled in the art. The fatty acid is typically covalently linked to the amine through an acyl bond. Synthesis typically starts from the C-terminus, to which amino acids are sequentially added using either a Rink amide resin (resulting in an —NH2 group at the C-terminus of the peptide after cleavage from the resin), or a Wang resin (resulting in an —OH group at the C-terminus). Accordingly, the present invention encompasses peptide amphiphiles having a C-terminal moiety that may be selected from the group consisting of —H, —OH, —COON, —CONH2, and —NH2.

In aqueous solutions, PA molecules self-assemble into cylindrical micelles that bury the lipophilic segment in their core and display the growth factor binding peptide on the surface. The structural peptide undergoes intermolecular hydrogen bonding to form beta sheets that orient parallel to the long axis of the micelle. The cylindrical micelles (also referred to as nanofibers) can form gels in water or various aqueous media under physiological conditions at concentrations ranging typically from 0.5 to 4 wt %.

To induce self-assembly of an aqueous solution of peptide amphiphiles, the pH of the solution may be changed (raised or lowered) or multivalent ions or charged polymers or other macromolecules may be added to the solution. Though not intending to be bound by theory, self-assembly is facilitated in the instant case by the neutralization or screening (reduction) of electrostatic repulsion between ionized side chains on the functional peptide segment. These cylindrical micelles formed by self-assembly can be viewed as fibrils or high-aspect-ratio nanostructures in which the functional peptide segment is repetitively displayed on the surface of the micelle.

B. Therapeutic Utilities and Methods of Use

As noted above, the present invention relates generally to novel VEGF-mimetic peptide-based scaffolds and their use in the context of therapeutic angiogenesis. Angiogenesis represents an excellent therapeutic target for combating diseases characterized by either poor vascularization or abnormal vasculature. Application of specific compounds that may induce the creation of new blood vessels in the body may help treat diseases associated with faulty or insufficient blood vessel formation such as ischemic disorders. For example, ischemic chronic wounds may be treated by a local expansion of blood vessels that bring new nutrients to the site and thus facilitate repair. Likewise, diminution of blood supply to vital organs associated with both coronary and peripheral artery diseases can be ameliorated through the production of new collateral vessels to overcome the ischemic insult. The promotion of angiogenesis is also desirable in the context of implanted tissue, biomaterial constructs, and other medical devices such as stents and prostheses. Foreign devices that include a means to promote vascular ingrowths achieve greater engraftment within the host tissue and thus are more biocompatible.

The mimetic PAs of the present invention and the self-assembled nanofiber scaffolds arising therefrom display angiogenic receptor-binding domains and thereby are able to activate receptors, induce pro-angiogenic effects, and trigger angiogenic responses in vivo. Accordingly, methods of treating diseases and disorders associated with poor circulation and blood vessel formation are contemplated by the present invention. For example, in one embodiment, the present invention provides a method of treating a patient suffering from such a disease or disorder that includes the step of administering a mimetic peptide amphiphile and/or nanofiber composition of the present invention to a target site, such as an occluded vessel or ischemic tissue. In one embodiment, a self-assembling peptide-amphiphile solution may be directly applied during a surgical procedure or delivered via a percutaneous injection into a target site within a human patient, wherein the self-assembled peptide-amphiphile gel organizes into a fibrillar scaffold or matrix. Intramuscular, subcutaneous, intravenous and intra-arterial administration routes are also contemplated. In an alternate embodiment, the self-assembling peptide solution may be mixed with cells in situ. Alternatively, the nanofibrous scaffold or matrix may be delivered in its assembled state, e.g., in the form of a gel or viscous solid. In yet another embodiment, cells such as endothelial cells may be suspended in a self-assembled peptide-amphiphile gel that is pre-formed into a matrix outside the body, which then can be implanted into a human patient.

Compositions of self assembled nanofibers or micelles can comprise hydrogels that may further include other therapeutic materials either encapsulated in or bonded to the hydrogel. These additional materials may include but are not limited to stem cells, growth factors, anti-inflammatories, anticoagulants, antibiotics, or combinations of these inside the nanofiber hydrogels. Preferably the additional materials are released to the surrounding tissue or cells through interaction with the extracellular receptors or by degradation of the nanofiber matrix.

One of skill in the art will readily recognize that a gel or solid composed of these nanofibers under physiological conditions of pH, temperature and tonicity affords the opportunity to utilize this material for a wide range of purposes and in a number of different potential biomedical and tissue engineering applications. To that end, the present invention provides a method of enhancing the biocompatibility of an interventional device or implant the includes the step of coating or otherwise applying the mimetic peptide amphiphiles and/or nanofiber compositions of the present invention to the desired target device. The peptide-amphiphiles of the present invention may be applied alone, or used in conjunction with other tissue engineering materials, either as a gel, solid, or liquid, to template cartilage tissue growth on one or more of the surfaces a device implanted in a human patient. Accordingly, in one embodiment, the present invention provides a method of treating a patient with tissue engineered material that includes the step of administering a peptide amphiphile composition to a target site on the patient in need of a tissue engineered material.

The present invention also contemplates methods and compositions for activating the differentiation and growth of vascular and endothelial cells. In particular, the present invention provides compositions composed of one or more self-assembling peptide amphiphiles (e.g., in solution) that generate (e.g., self-assemble into) nanofibers that display mimetic peptides on the surface and interact with respective pro-angiogenic receptors, thereby enabling the transmission of signals which lead to the transcription of several genes that promote vascular and endothelial regeneration. In addition to peptide amphiphiles and nanofibrous matrices assembled therefrom, the present invention also contemplates pharmaceutical compositions composed of one or more of such amphiphiles or matrices; all find use in both research and clinical (e.g., therapeutic) settings.

Hereinafter, the present invention is described in more detail by reference to the Examples. However, the following materials, methods and examples only illustrate aspects of the invention and in no way are intended to limit the scope of the present invention. As such, methods and materials similar or equivalent to those described herein can be used in the practice or testing of the present invention.

### EXAMPLES

The following non-limiting examples and data illustrate various aspects and features relating to the amphiphile compounds, nanofibers, gels, compositions and/or methods of the present invention, including the self-assembly of VEGF mimetic peptide amphiphiles and corresponding delivery thereof, as are available through the methodologies described herein. In comparison with the prior art, the present methods, compounds and compositions provide results and data that are surprising, unexpected and contrary thereto. While the utility of this invention is illustrated through the use of several amphiphilic compounds and components thereof, it will be understood by those skilled in the art that comparable results are obtainable with various other amphiphile compounds and/or components, as are commensurate with the scope of this invention.

### Example 1

### Design and Synthesis of a VEGF-Mimetic PA

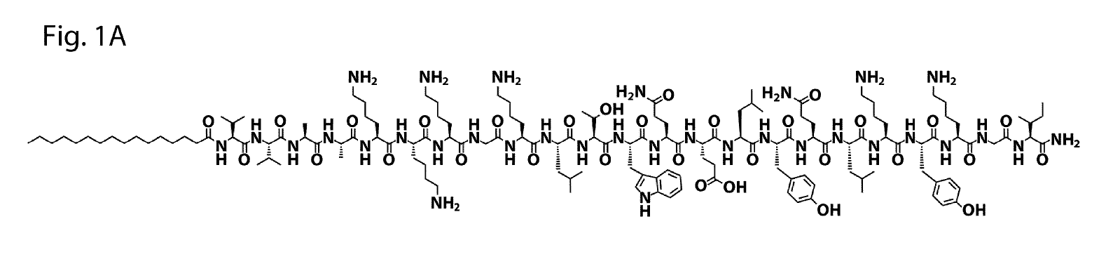

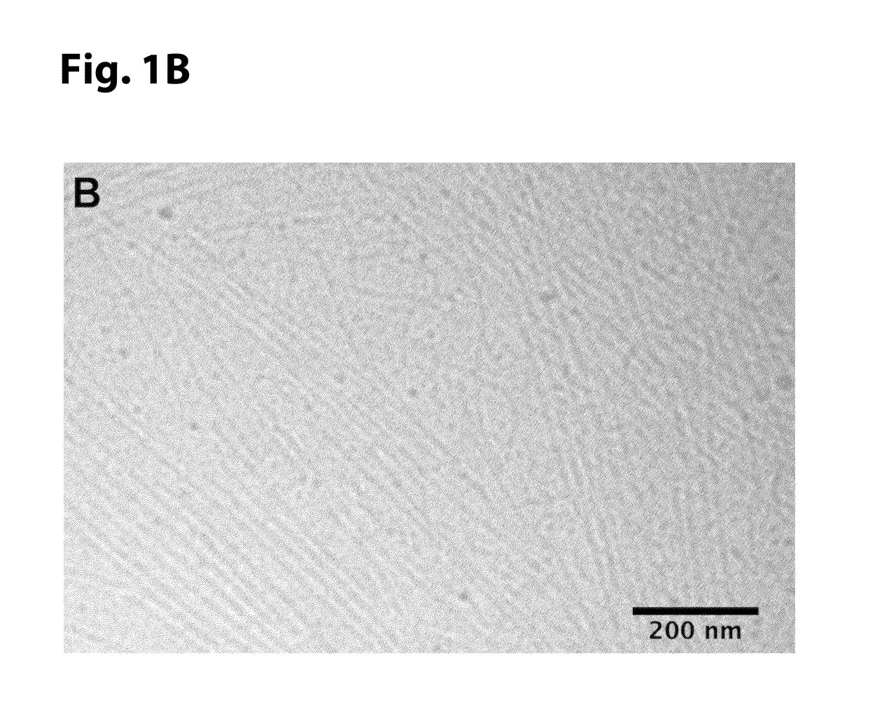

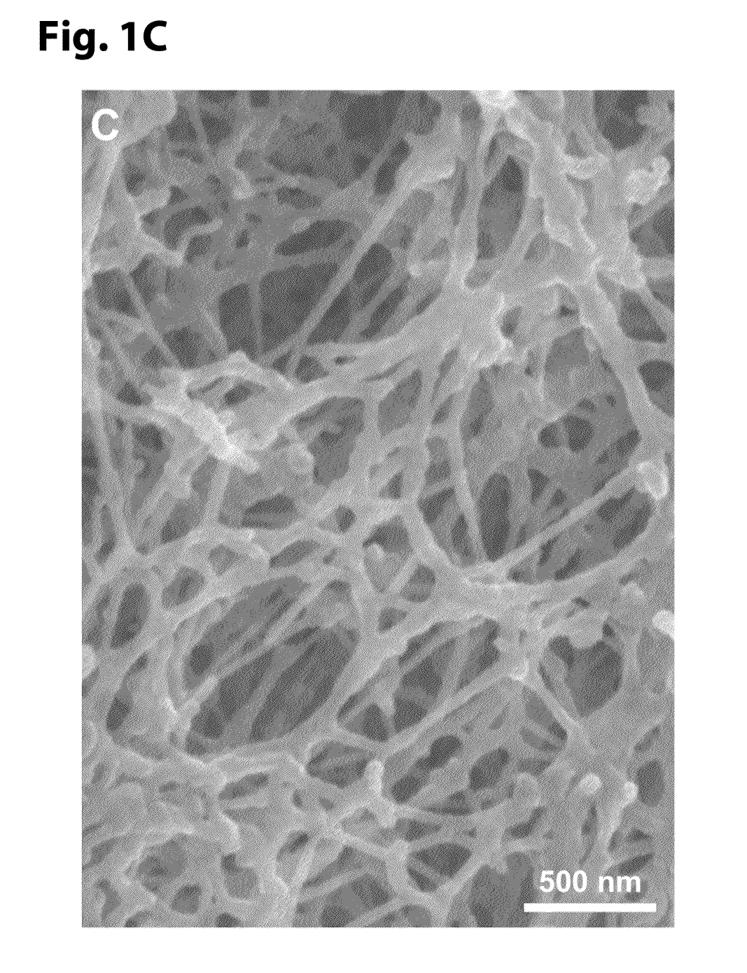

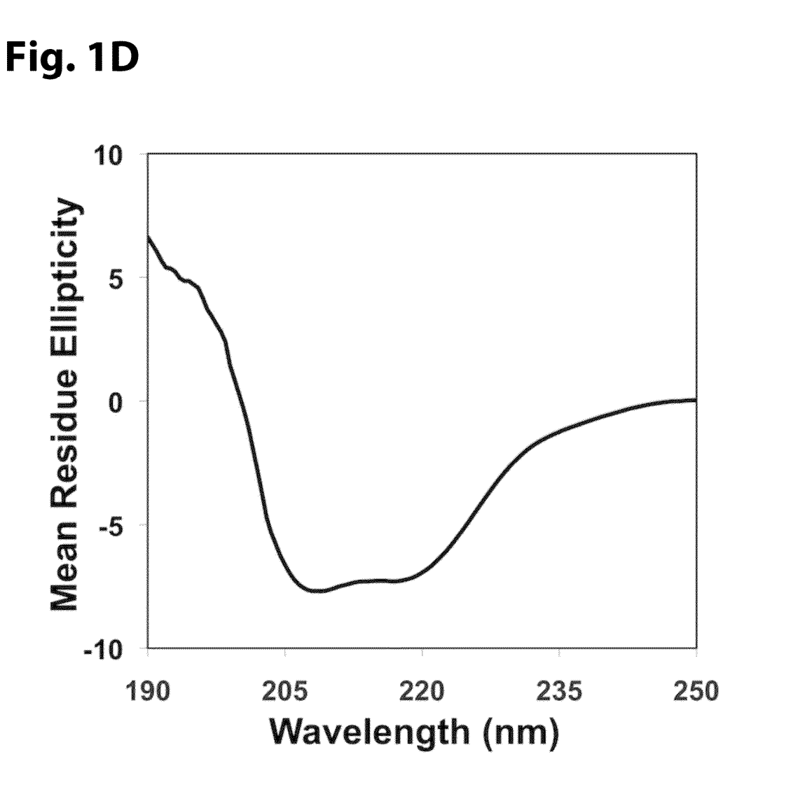

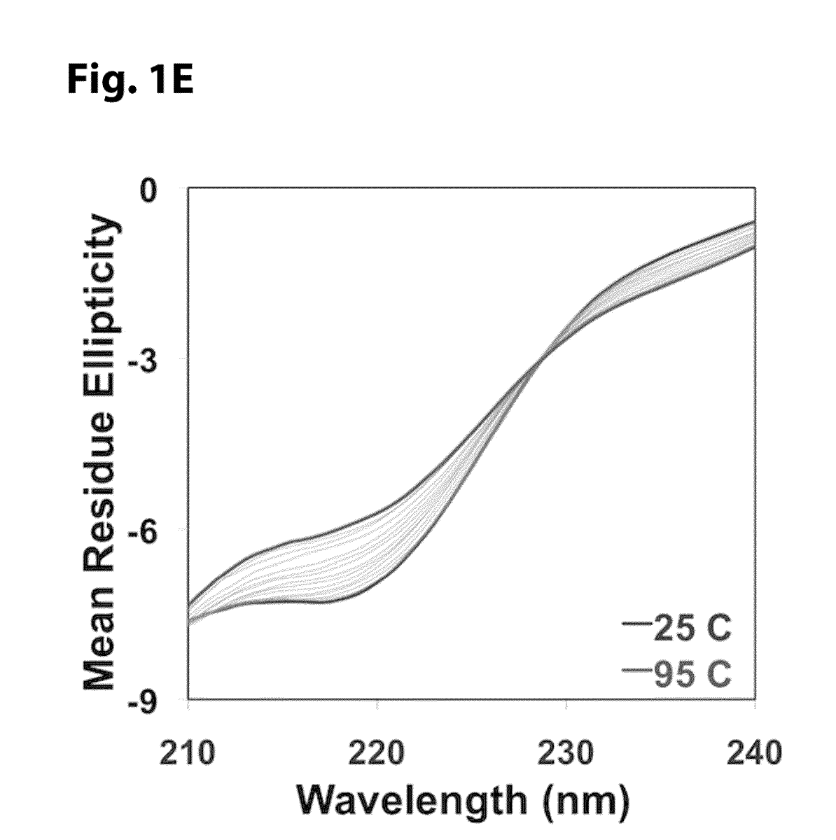

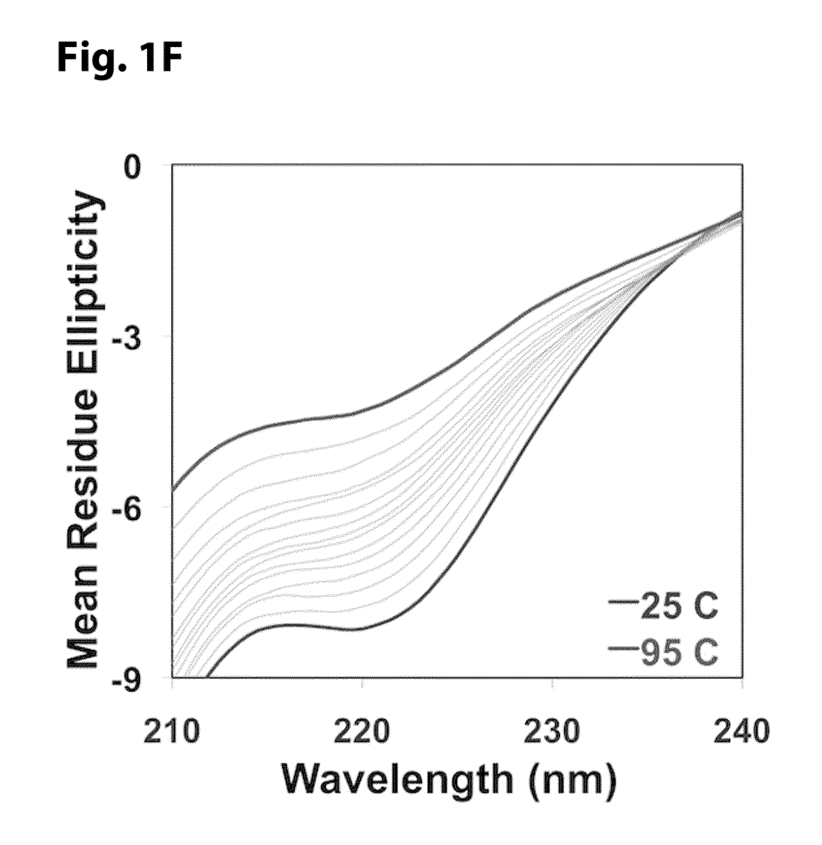

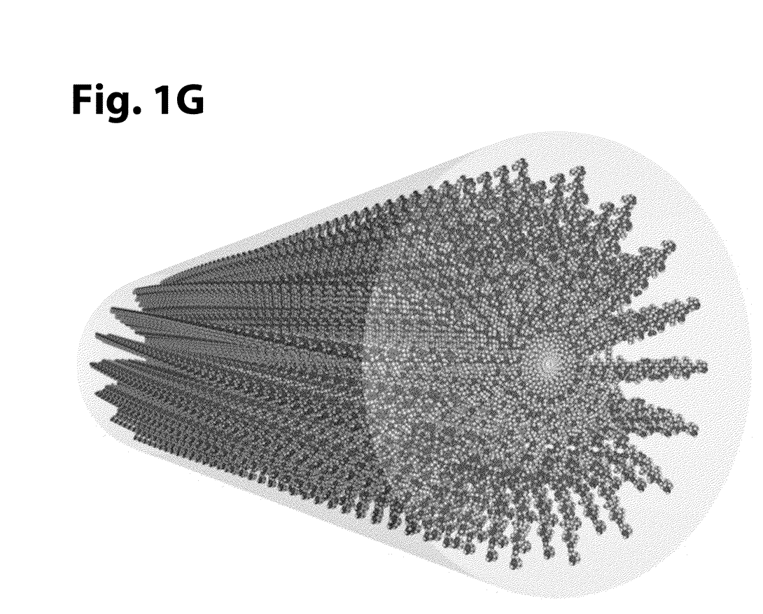

The VEGF-PA (FIG. 1A) utilized in the present examples was designed to display on surfaces of nanostructures a peptide sequence that mimics VEGF, namely KLTWQELYQLKYKGI-NH2 (SEQ ID NO: 2) [12]. A glycine (G) spacer was covalently attached to the N-terminus of this peptide, followed by a triple lysine (K3) sequence intended to improve solubility and trigger electrolyte-driven self-assembly. The K3G sequence was followed by a V2A₂ β-domain and a C16 acyl chain to promote self-assembly into cylindrical nanostructures through intermolecular hydrogen bonding and hydrophobic collapse upon electrolyte screening of charged residues (FIG. 1G).

Stabilization of the bioactive conformation of the VEGF-mimetic peptide by conjugation to a PA could be advantageous to enhance the potency of the epitope. To demonstrate the improvement obtained a VEGF-PA and epitope-only peptide control were synthesized. In addition, as a nanostructure control for the VEGF-PA, a mutant PA control was synthesized.

This control was prepared by modifying four specific residues reported to be near the peptide-receptor binding interface with structurally different amino acids. The three PAs synthesized have the following amino acid sequences were synthesized: CH3(CH2)14C(O)— VVAAKKKGKLTWQELYQLKYKGI-NH2 (SEQ ID NO: 3, termed herein “VEGF-PA”), Ac-KLTWQELYQLKYKGI-NH2 (SEQ ID NO: 4, termed herein “VEGF peptide”) and CH3(CH2)14C(O)—VVAAKKKGKLTAQELVFLKVKGI-NH2 (SEQ ID NO: 5, termed herein “mutant PA”). The structure of VEGF-PA is shown in FIG. 1A.

All peptides were synthesized by standard solid phase Fmoc chemistry. Fmoc-protected amino acids, MBHA rink amide resin, and HBTU were purchased from NovaBiochem and all reagents were purchased from Mallinckrodt. The resulting product was purified using standard reversed-phase HPLC. The purity and accurate mass for each PA was verified using LC/MS on an electrospray ionization quadripole time-of-flight mass spectrometer (Agilent). Additionally, peptide content analysis was performed on the purified product (Commonwealth Biotechnologies, Inc. Richmond, Va.) to ensure concentration accuracy and consistency for all experiments.

### Example 2

### Structural Characterization of a VEGF-Mimetic PA

Cryogenic TEM was performed on a JEOL 1230 microscope with an accelerating voltage of 100 kV. A Vitrobot Mark IV equipped with controlled humidity and temperature was used for plunge-freezing samples. A small volume (7 μl) of 0.05 w/v % VEGF-PA dissolved in 0.5×PBS was deposited on a copper TEM grid with Quantifoil support film (Electron Microscopy Sciences) and held in place with tweezers mounted to the Vitrobot. The specimen was blotted in 90-95% humidity and plunged into a liquid ethane reservoir cooled by liquid nitrogen. The vitrified samples were transferred into liquid nitrogen and inserted into a Gatan 626 cryo-holder through a cryo-trarisfer stage. Samples were imaged using a Hamamatsu ORCA CCD camera.

SEM was performed using a Hitachi S4800 scanning electron microscope with a 5 kV accelerating voltage. To prepare samples for imaging, VEGF-PA was dissolved at 1.5 w/v % in water and mixed with 10 mM Na2HPO4 to induce hydrogelation at 1 w/v %. The sample was fixed in 2% glutaraledyde and 3% sucrose in PBS for 30 minutes at 4° C. followed by sequential dehydration in ethanol. It was then dried at the critical point and coated with 7 nm OsO4 prior to imaging.

Cryogenic TEM of the PAs in pure water reveals the formation of self-assembled high aspect ratio cylindrical nanostructures (FIG. 1B). At a concentration later used for therapeutic assessments and in the presence of divalent counterions, these VEGF-PA nanostructures entangled to form nanofiber gels, observed by SEM (FIG. 1C).

Circular dichroism (CD) was performed on a Jasco J-815 CD spectrophotometer complete with Peltier sample holder for precise temperature control. Samples were analyzed at 0.15 mM in water, correcting for absolute peptide content. Measurements were collected over a wavelength range of 260-180 nm with a step size of 0.5 nm and 5 total accumulations for each scan. For thermal denaturation studies, samples were heated from 2° C. to 95° C. at a step of 5° C. and held for 10 minutes prior to each reading.

The bioactive conformation of this peptide sequence is known to have a α-helical secondary structure [12, 13]. CD of the VEGF-PA in water reveals, in fact, a signal characteristic of an a-helix (FIG. 1D). The greater conformational stability of this a-helix for the epitope within PA molecules (FIG. 1E) compared to the peptide alone (FIG. 1F) is indicated by a smaller change in the 220 nm a-helical CD signature when increasing temperature from 25 to 95° C. Conjugation to an alkyl tail is known to stabilize an a-helical peptide [28], which could explain the increased thermal stability of the a-helical epitope in PA molecules.

### Example 3

### VEGF-PA Specifically Activates VEGF Receptors

VEGF signal transduction is initiated by multiple tyrosine phosphorylation events on the intracellular domain of its receptors [29]. In order to determine if the VEGF-PA specifically signals in a manner consistent with VEGF, human umbilical vein endothelial cells (HUVEC) were stimulated with VEGF-PA and a sandwich ELISA was performed on cell lysates to quantify the amount of phosphorylated VEGF receptor 1 (VEGFR1 or Flt-1) or phosphorylated VEGF receptor 2 (VEGFR2 or KDR), the two primary VEGF receptors implicated in its angiogenic signaling.

Human umbilical vein endothelial cells (HUVECs) and complete endothelial cell growth media (EGM) were purchased (Genlantis, San Diego, Calif.), passaged two times following receipt, and cryo-preserved in media with 5% DMSO. Cells were thawed as needed and grown to confluence in a 75 mm2 flasks (VWR Falcon) prior to plating for experiments.

Phosphorylation of both VEGFRI and VEGFR2 was assayed using commercially available assay kit (R&D Systems, Minneapolis, Minn.) following the recommended protocol. First, confluent HUVECs plated in 24 well plates were starved for 5 hours in serum-free defined media (SFDM, Genlantis) specifically designed to maintain cells in a growth factor-free setting for growth factor signaling and metabolic assays. Following starvation, the various treatments were dissolved in SFDM to a concentration of 1 μM. Native VEGF (100 ng/ml) diluted in SFDM was used according to assay recommendations in order to serve as an internal assay control for VEGF phosphorylation patterns and a group was treated with plain SFDM as a baseline control. Cells were exposed to treatment for 2 minutes. To evaluate the time-course of phosphorylation the same protocol was followed and cells were exposed to VEGF-PA for 0, 1, 3, 5, 10, 15, 20, 25, 30, 40, 50, and 60 minutes.

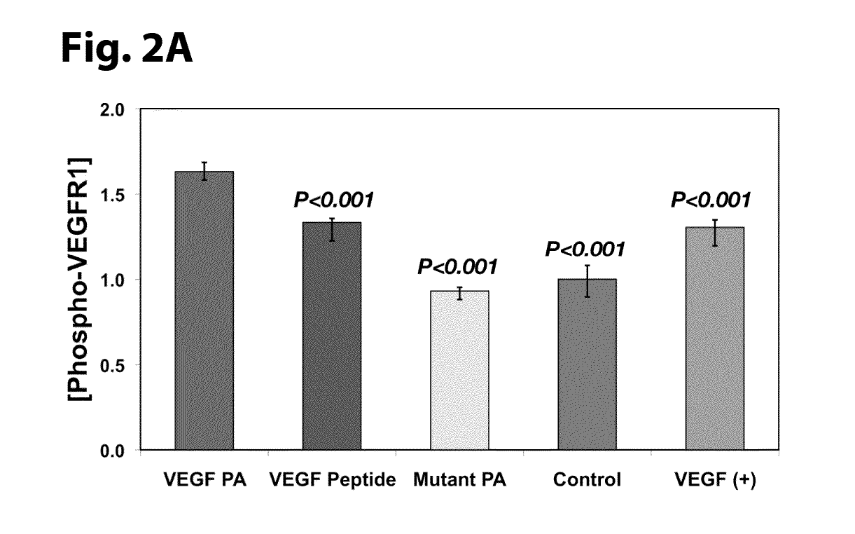

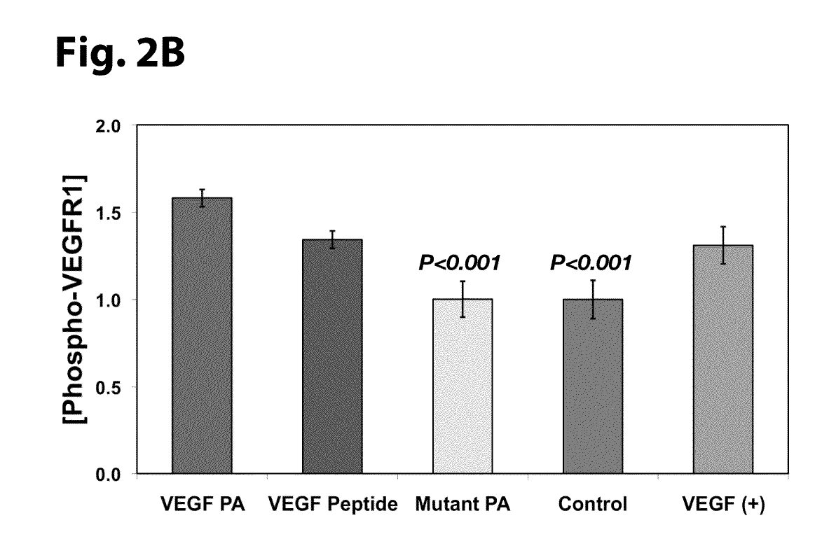

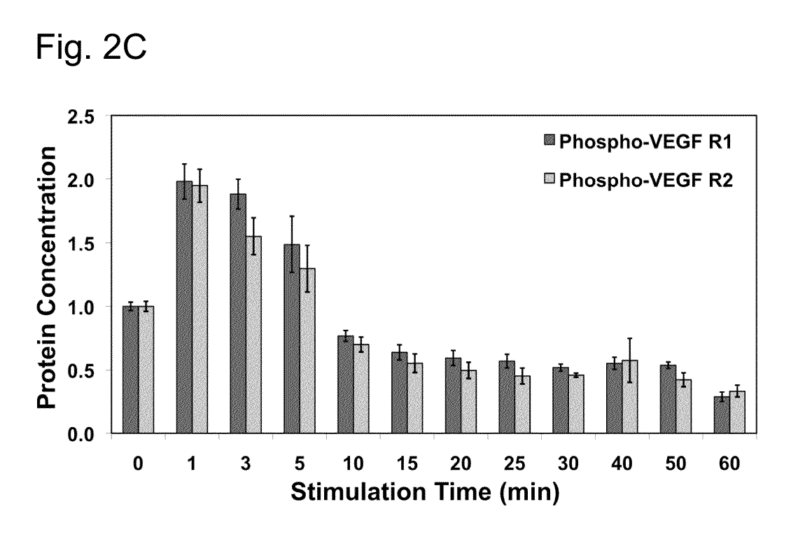

Stimulation with the VEGF-PA resulted in an amount of phosphorylated VEGFR1 (FIG. 2A, n=6/group) that was 1.632±0.054 times greater than an untreated control group (1.000±0.080, P<0.001), showing a significant enhancement in receptor phosphorylation. Interestingly, the level of phosphorylation for the VEGF-PA was significantly greater than for an identical concentration of the bioactive VEGF epitope peptide (1.335±0.024, P<0.001), a positive control of VEGF protein (1.306±0.044, P<0.001), and mutant PA (0.931±0.022). Both the bioactive peptide and the VEGF protein revealed significantly greater phosphorylation than untreated samples.

For VEGFR2 phosphorylation (FIG. 2B, n=6/group), the VEGF-PA (1.581±0.050) again demonstrated phosphorylation levels significantly greater than that for an untreated control (1.000±0.109, P<0.001). The value for the VEGF peptide (1.343±0.050) and the positive control of VEGF (1.311±0.106) were again less than those observed for the VEGF-PA, however these differences were not significant for VEGFR2. The mutant PA (1.001±0.103, P<0.01) again demonstrated no effect on VEGFR2 phosphorylation, establishing this as an ideal material control for our VEGF-PA in other experiments. The VEGF peptide signals similarly to VEGF for both receptors, confirming reports from the discovery of the epitope [12].

Looking at the effect of VEGF-PA stimulation over time (FIG. 2C, n=4/time), we observe an initial rise in the levels of both phosphorylated VEGFR1 and VEGFR2, followed by a decrease beginning at 10 minutes of stimulation to levels below the baseline of an untreated control. The response and timeframe of signaling by VEGF-PA is consistent with the known response for VEGF receptor activation followed by the subsequent cleaving of phosphates and ubiquitination of the receptor [30].

Overall, phosphorylation of VEGFR1 and VEGFR2 by the VEGF-PA of the instant examples is indicative of VEGF-pathway specific signaling at the receptor level. The temporal pattern of phosphorylation induced by this VEGF-PA is also consistent with that reported for VEGF.

### Example 4

### VEGF-PA Promotes Angiogenic Function In Vitro

VEGF signaling is known to enhance, among other cellular functions, the proliferation, survival and migration of endothelial cells [3, 29]. In vitro studies were performed using HUVECs, a primary endothelial cell that is VEGF-responsive and frequently used as a model endothelial cell for angiogenesis studies in vitro [31].

To evaluate proliferation, HUVECs were plated at 5000 per well in a 96 well plate. Four hours after plating, EGM was exchanged for fresh EGM supplemented with 1 μM of VEGF-PA, VEGF peptide, or mutant PA (n=8/group). Additionally, unsupplemented growth media was used as a control. After 48 hours, cell number was quantified using CyQUANT-NF (Invitrogen) and a standard fluorescent microplate reader. Cell number is expressed relative to the group treated with unsupplemented growth media.

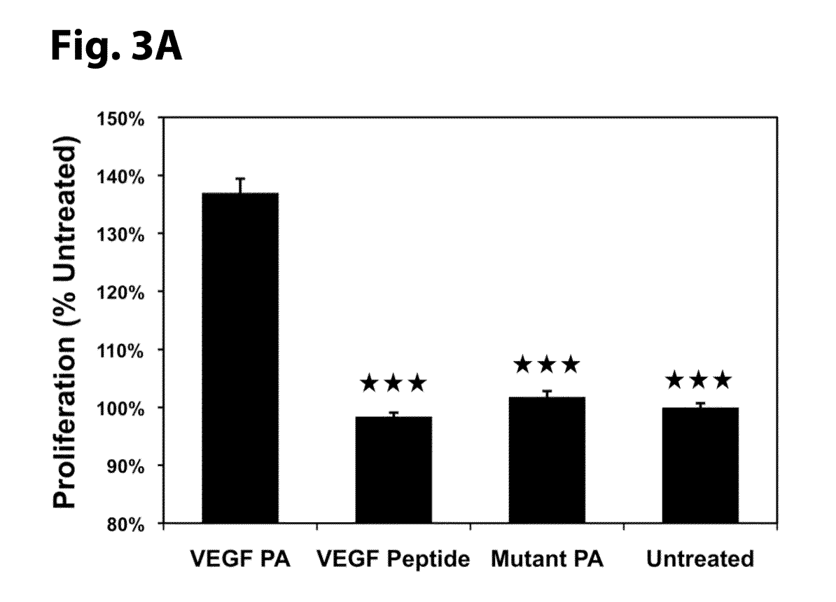

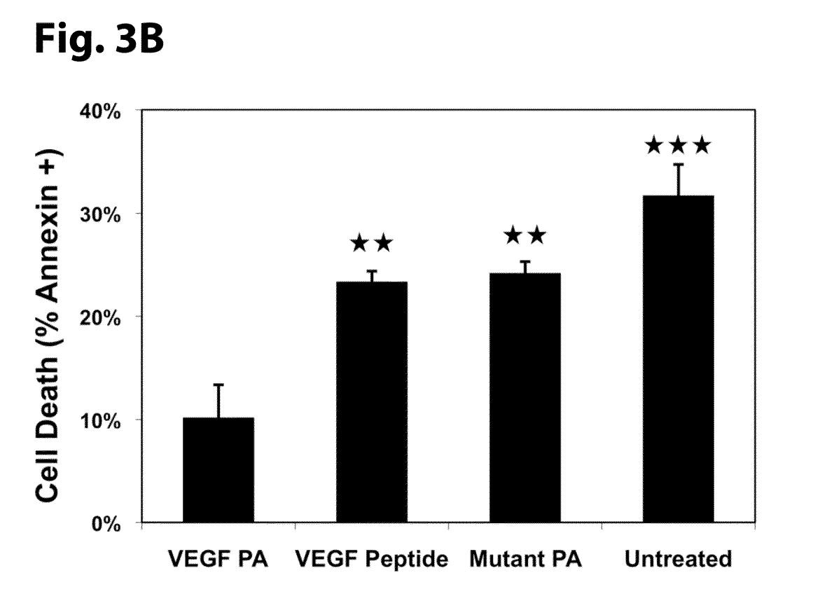

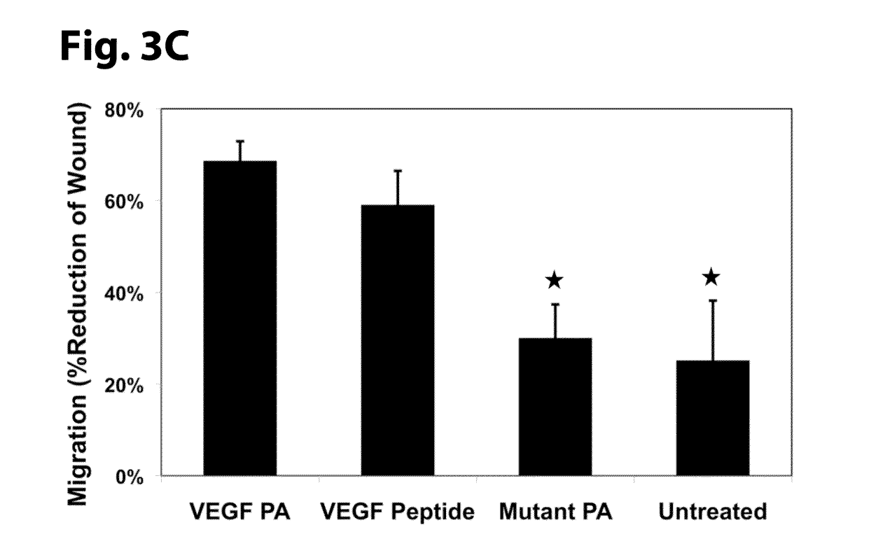

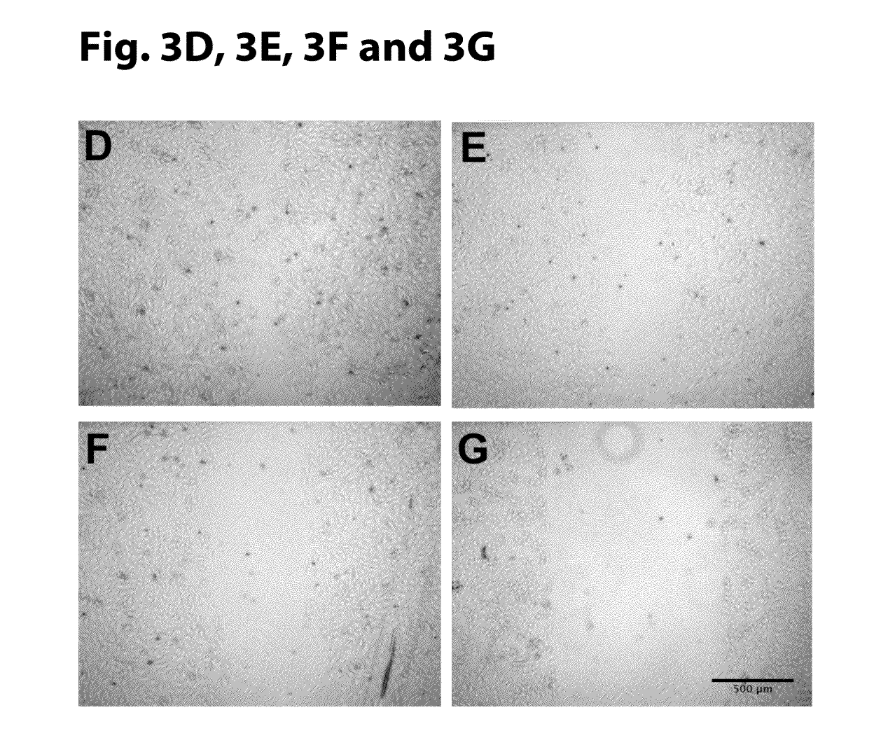

Assessing the effects of the designed VEGF-PA on HUVEC proliferation (FIG. 3A, n=8/group), it was determined that stimulation with the VEGF-PA significantly increases proliferation (137.0%±2.4%) relative to an untreated control (100.0%±0.8%, P<0.001). VEGF-PA also significantly enhanced proliferation relative to treatment with identical epitope molar concentrations of the VEGF peptide (98.4%±0.7%, P<0.001) and the mutant PA (101.8%±1.4%, P<0.001), respectively. Incubation with either the VEGF peptide or the mutant PA did not significantly effect proliferation relative to an untreated control.

To evaluate cell survival, endothelial basal media (EBM) without growth factors (Lonza) was used to induce a serum-starvation. Cells were plated in 12 well plates and grown to confluence with standard growth media. Cells were then washed twice with PBS and then treated with EBM containing 1 μM of VEGF-PA, VEGF peptide, or mutant PA along with an untreated control receiving only EBM. Cells were grown in these conditions for 24 hours. Survival was quantified using Annexin V: PE staining with 7-AAD vital staining (BD Biosciences) following provided assay instructions and analyzed on a DakoCytomation CyAn. Survival was assessed by determining the fraction of cells that were apoptotic (Annexin V-PE positive).

Cell survival was assessed after challenging HUVECs with serum-starvation for 24 hours and measuring the fraction of apoptotic cells, as described above (FIG. 3B, n=6/group). Cells starved in the presence of VEGF-PA (10.2%±3.2%) survived significantly better than an untreated group (31.7%±3.0%, P<0.001). The VEGF-PA group also survived significantly better than did cells incubated with identical concentrations of either the VEGF peptide (23.4%±1.1%, P<0.01) or mutant PA (24.2%±1.1%, P<0.01). Neither the VEGF peptide nor the mutant PA group significantly enhanced survival relative to the untreated case, indicating the pro-survival effect of VEGF-PA is not solely due to the presence of additional peptidic metabolites in the media.

To evaluate cell migration, endothelial basal media (EBM) without growth factors (Lonza) was supplemented with 0.5% FBS in order to prevent apoptosis but not promote significant migration or proliferation. Cells were grown to confluence in a 12 well plate and a 1 ml pipette tip was used to create a denuded scratch and the surface was washed twice with PBS to remove detached cells. The average scratch width at the initial time-point was 926.3±103.0 Markings were placed on the underside of the plate to ensure the same region of the scratch was recorded in each image. Following scratch creation, the cells were treated with 1 μM VEGF-PA, VEGF peptide, mutant PA in EBM with 0.5% FBS. The total pixel area of the scratch at the initial time-point was recorded using ImageJ analysis software, and the percent migration was determined from the reduction in denuded area at 18 hours.

The effect on cell migration was assessed using a scratch assay, as describe above, (FIG. 3C, n=6/group) and the reduction in the denuded area was measured. For the group stimulated with VEGF-PA, the reduction in scratch wound size (68.7%±4.2%) was significantly greater than for an untreated control (25.2%±13.0%, P<0.05) and also for the group stimulated by the mutant PA (30.0%±7.3%, P<0.05). However, treatment with the VEGF peptide (52.5%±4.1%) did not significantly enhance migration relative to the untreated control group.

Overall, the robust effects of VEGF-PA on HUVEC proliferation, survival, and migration in vitro are consistent with established VEGF-specific endothelial cell functional activity. This effect on endothelial cell function, combined with the above-noted demonstration of VEGF-specific receptor phosphorylation, supports the VEGF-mimetic activity in vitro of the VEGF-PA nanofibers. The VEGF peptide, meanwhile, did not show similar effects on endothelial cell function in vitro, even though it demonstrated an increase in phoshorylation as compared to untreated controls in the ELISA studies. However, phosphorylation studies were performed for only a single phosphorylation cycle so it is possible that the peptide does not retain potency over longer times in culture. Alternatively, it is possible that the additional phosphorylation induced by VEGF-PA as compared to the VEGF peptide seen for one phosphorylation cycle leads to an overall cumulative effect on receptor activation over the time-course of many phosphorylation cycles afforded by these longer in vitro experiment times.

### Example 5

### VEGF-PA Induces Angiogenesis In Vivo

An established assay reflecting in vivo angiogenesis, the chicken chorioallantoic membrane (CAM) assay, was used to evaluate the angiogenic potential of the VEGF-PA of the present examples. This assay utilizes the extra-embryonic allantois, a tissue derived from the mesoderm that develops into a densely vascularized membrane. A common deviation from the traditional assay is to remove the shell and conduct the assay on a shell-less embryo, termed the shell-less CAM assay, as we have performed here [51, 52]. Fertilized white leghorn chicken eggs (Phil's Fresh Eggs, Forreston, Ill.) were received and cultured in a temperature controlled, humidified egg incubator. On embryonic day 3, eggs were cracked within a sterile tissue culture hood into round 100 mm petri dishes. Fertilized embryos were then transferred to a water-jacketed CO2 incubator, set to 37.5° C., 1% CO2 and 100% relative humidity. On embryonic day 10, the material treatment was dissolved at 2 mM in PBS, evaporated onto the surface of a 5 mm round glass cover slip, and placed facing down on top of the CAM (n=16/group). Digital images were captured through the eyepiece of a Nikon stereomicroscope and vessel density was quantified by the number of intersections of vessel structures with the edge of the cover slip and expressed relative to the initial time-point. Images were captured daily, beginning at embryonic day 10 and culminating on embryonic day 13, the standard range over which the CAM assay is performed. Additionally, images were assessed qualitatively for morphological differences in the CAM vasculature including spoking, branching and leakage.

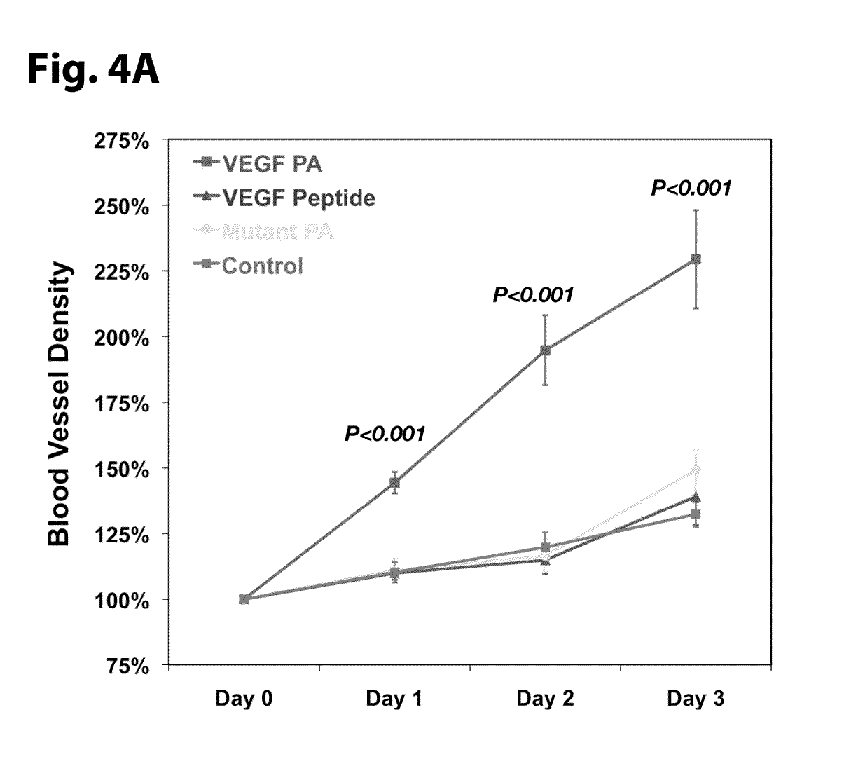

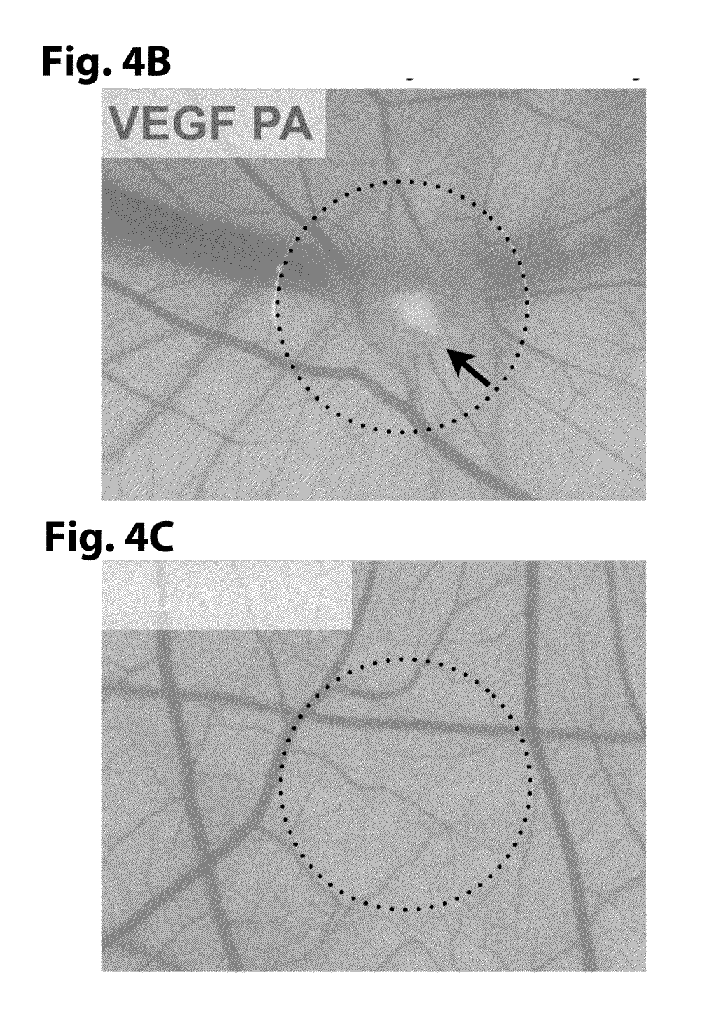

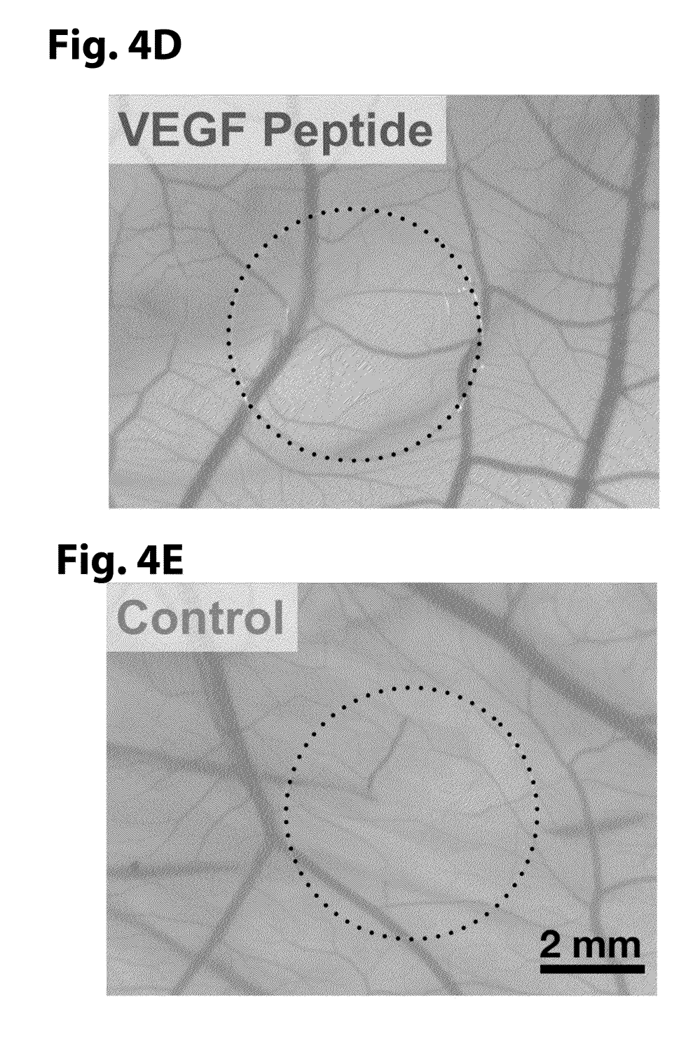

When VEGF-PA was coated onto a glass coverslip and applied to the CAM (FIG. 4), we saw a significant increase in the blood vessel density over the next 3 days of follow-up compared to groups treated with the peptide control, mutant PA, or saline. By three days following treatment, CAM treated with VEGF-PA showed a 229%±19% increase in blood vessels relative to the initial value. For comparison, this was significantly greater (P<0.001) than CAM treated with the VEGF peptide (139%±11%), mutant PA (149%±8%) or saline (132%±5%). This increase suggests a strongly angiogenic response attributable to treatment with the VEGF-PA. This can be visualized in the density of blood vessels at the point of CAM stimulation with VEGF-PA in addition to observations of vascular remodeling and leakage and the spoke-like pattern radiating from the center of the coverslip where the material was applied. Representative images from the various controls do not display a similar effect. This assay confirms the angiogenic properties of the VEGF-PA of the instant examples using an in vivo model, and reinforces the pro-angiogenic findings from our in vitro evaluation.

### Example 6

### VEGF-PA Enhances Repair of Ischemic Hind-Limb Tissue

In order to assess the therapeutic potency of VEGF-PA for ischemic tissue repair, we chose the hind-limb ischemia (HLI) model, an established model for critical tissue ischemia. For the HLI procedure, eight week old male FVB wild-type mice (Charles River) were anesthetized by intraperitoneal injection of avertin (125 mg/kg). By means of a dissecting microscope, the femoral nerve was carefully separated from the vessel bundle. The right femoral artery was ligated and excised, including all superficial and deep branches [53]. Critical limb ischemia was immediately verified by laser Doppler imaging (LDPI, MoorLDI-SIM, Moor Instruments, Ltd) to ensure the ratio (ischemic/non-ischemic limb) was ≦0.20. At postoperative day 3, outliers with low ischemia were triaged based on LDPI (ratio ischemic/nonischemic 0.30) as were outliers with extreme ischemia (necrotic demarcation of entire limb), determined by macroscopic evaluation. After triage, all remaining mice were treated by a single transcutaneous intramuscular injection (25 μl) of 2 mM VEGF-PA, VEGF peptide, mutant PA, or saline (PBS) as control. For follow-up, animals underwent reevaluations with LDPI at postoperative day 7, 14, and 28 before animals were sacrificed (CO, asphyxia) for tissue harvest at day 28. At each time-point, tissue perfusion was measured via LDPI, measuring blood flow in both the ischemic and non-ischemic limb and reporting results as the ratio of these two measurements. All LDPI measurements were taken on a 37° C. heating pad to control body temperature. In addition, motor function and tissue damage was semi-quantitatively assessed on post-operative day 7, 14, 21 and 28 by established scoring systems. Tissue damage in the ischemic limb (limb salvage score) was graded as full recovery (grade 6), minor necrosis or nail loss (grade 5), partial toe amputation (grade 4), total toe amputation (grade 3), partial/total foot amputation (grade 2) or partial/total limb amputation (grade 1) (modified from [54]). Limb motor function was graded as unrestricted (grade 5), no active use of toe(s) or spreading (grade 4), restricted foot use (grade 3), no use of foot (grade 2) or no use of limb at all (grade 1) (modified from [55]). Finally, walking capacity was measured via a Rota Rod apparatus. The rotational velocity was steadily increased and time at which the mouse failed to keep up with the treadmill was recorded. The mean of two assessments was used as for failure time for each animal.

At day 28, muscle tissue from the ischemic limb was harvested, fixed in methanol, paraffin-embedded, and cross-sectioned (6-μm) for histological immunostaining. Briefly, sections were blocked with 10% donkey serum (30 min, RT). Primary antibodies were diluted in PBS containing BSA, and applied to tissue slices for 2 hrs at 37° C. Sections were stained for CD31, an endothelial-specific marker, using rat anti-CD31 antibodies (BD Pharmingen Inc) and smooth-muscle α-actin (αSMA), a vascular smooth muscle marker, using rabbit-anti-αSMA (Sigma-Aldrich Co.). For immunofluorescent detection, primary antibodies were resolved with Alexa-Fluor-conjugated secondary antibodies (Invitrogen Corporation, Carlsbad, Calif., USA dilution) and nuclei were counterstained with DAPI (Research Organics). Slides were imaged using fluorescent microscopy (Zeiss), and CD31+capillary forms and CD31+/sMA+]mature microvessels/arterioles were quantified in 3 separate high-power fields (20×) from 3 independent sections in each animal (9 images/animal).

All error bars indicate the standard error of the mean. Differences between groups were determined using a one-way analysis of variance (ANOVA) with a Bonferroni multiple comparisons post-hoc test using GraphPad InStat v3.0b.

The murine hind-limb ischemia model was selected to evaluate the potential of the VEGF mimetic PA nanostructures as a therapy for ischemic cardiovascular diseases. VEGF-PA or control treatments were administered by an intramuscular injection three days following the induction of critical ischemia by ligation and excision of the right femoral artery and all superficial and deep branches, as described above. Animals were assessed serially throughout the study for motor function and limb salvage and Laser Doppler perfusion imaging (LDPI) was used to assess tissue perfusion. At the end of the study, exercise endurance was recorded on a Rota Rod treadmill and tissue was harvested for immunohistological staining of capillaries to observe effects on the microcirculation.

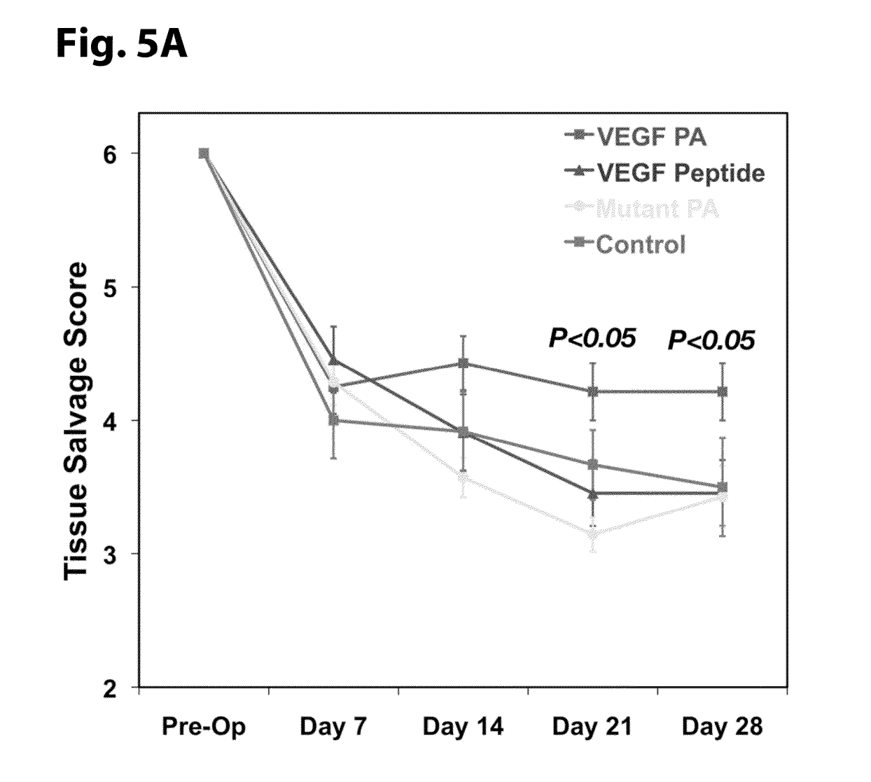

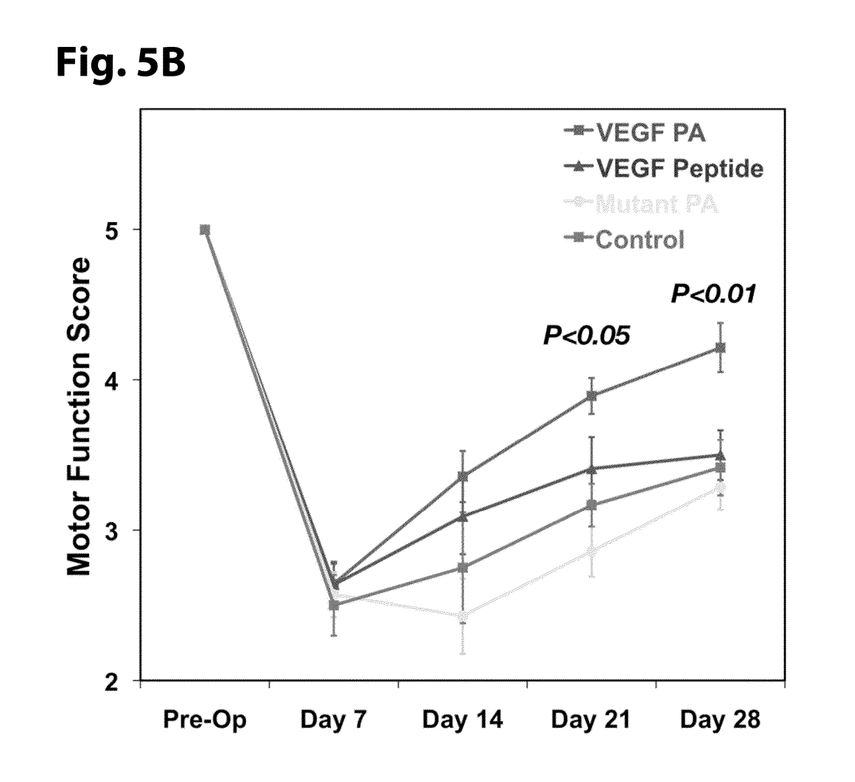

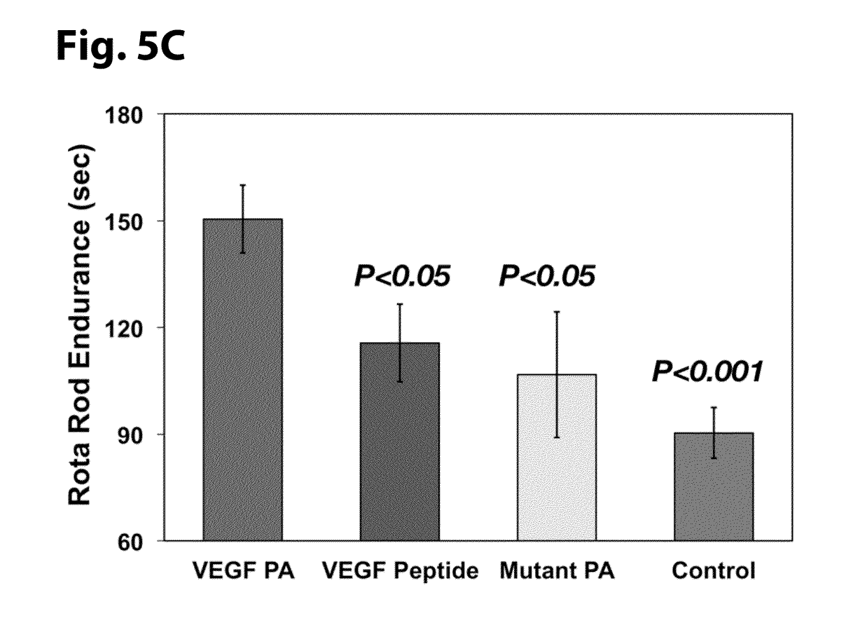

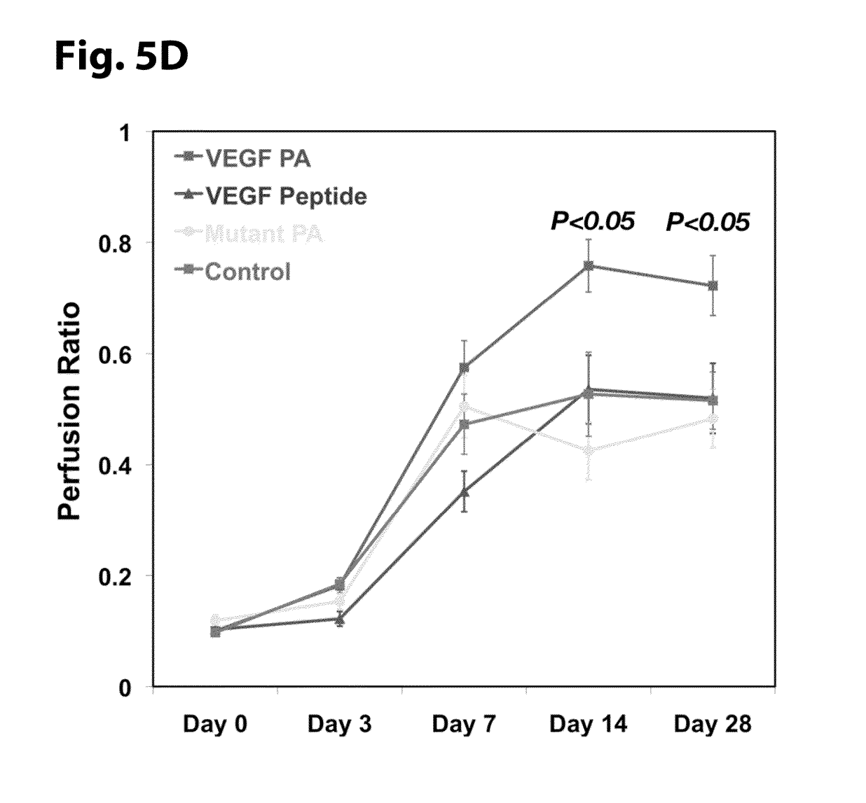

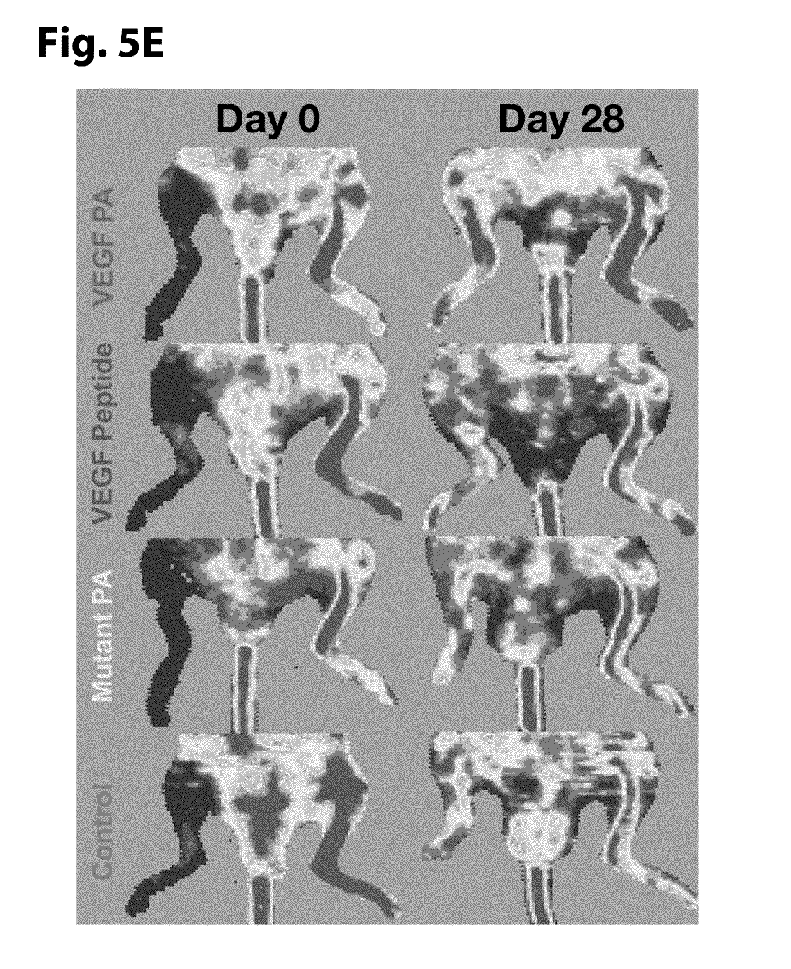

To assess functional recovery after critical hind-limb ischemia, animals were assessed for limb salvage and limb motor function via established semi-quantitative scoring methods. In terms of tissue necrosis and amputation of ischemic limb, significant improvement was observed for tissue salvage (i.e. less necrosis) in animals treated with VEGF-PA (FIG. 5A) at both day 21 (4.21±0.21) and day 28 (4.21±0.21) as compared to animals treated with VEGF peptide (day 21: 3.45±0.25, P<0.05; day 28: 3.45±0.25, P<0.05), mutant PA (day 21: 3.14±0.26, P<0.05; day 28: 3.43±0.37, P<0.05), and saline (day 21: 3.67±0.13, P<0.05; day 28: 3.50±0.23, P<0.05).

When scoring animals based on active limb motor function (FIG. 5B), a significant effect was again observed for treatment with the VEGF-PA at day 21 (3.89±0.12) and day 28 (4.21±0.16) as compared to treatment with the VEGF peptide (day 21: 3.41±0.21, P<0.05; day 28: 3.50±0.17, P<0.01), mutant PA (day 21: 2.86±0.14, P<0.01; day 28: 3.29±0.18, P<0.01) and saline control (day 21: 3.17±0.17, P<0.01; day 28: 3.42±0.15, P<0.01). This assessment of limb use suggests that treatment with the VEGF-PA leads to a more functional phenotype over time. This semi-quantitative observation was further supported by another measure to assess motor function. At 28 days, animals were subjected to walking endurance testing using a Rota Rod treadmill with progressively increasing speed (FIG. 5C). Animals treated with VEGF-PA walked significantly longer prior to failure (150.5±9.5 seconds) than animals treated with the VEGF peptide (115.6±10.9 seconds, P<0.05), mutant PA (106.7±17.6 seconds, P<0.05) and saline (90.4±7.2 seconds, P<0.001).

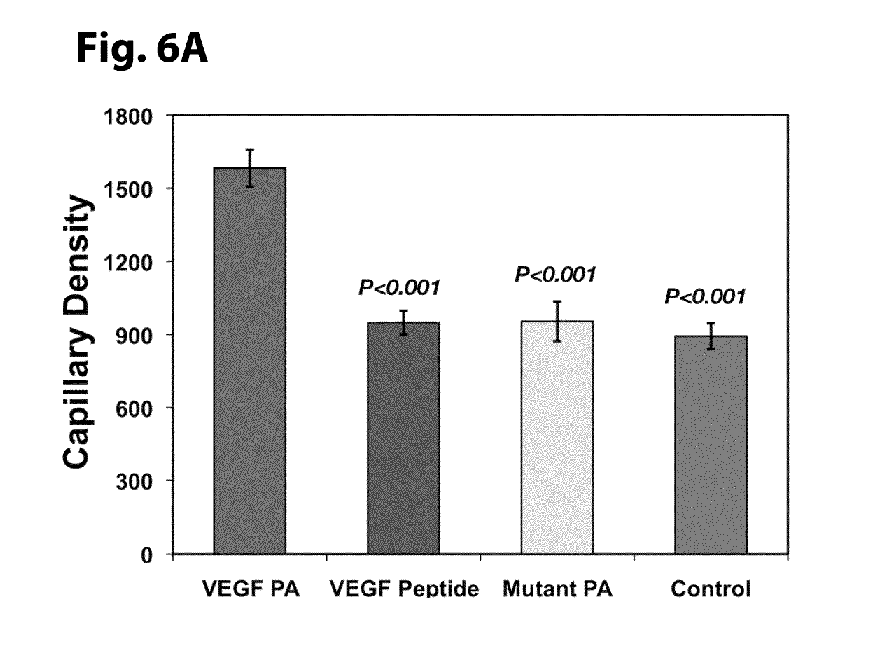

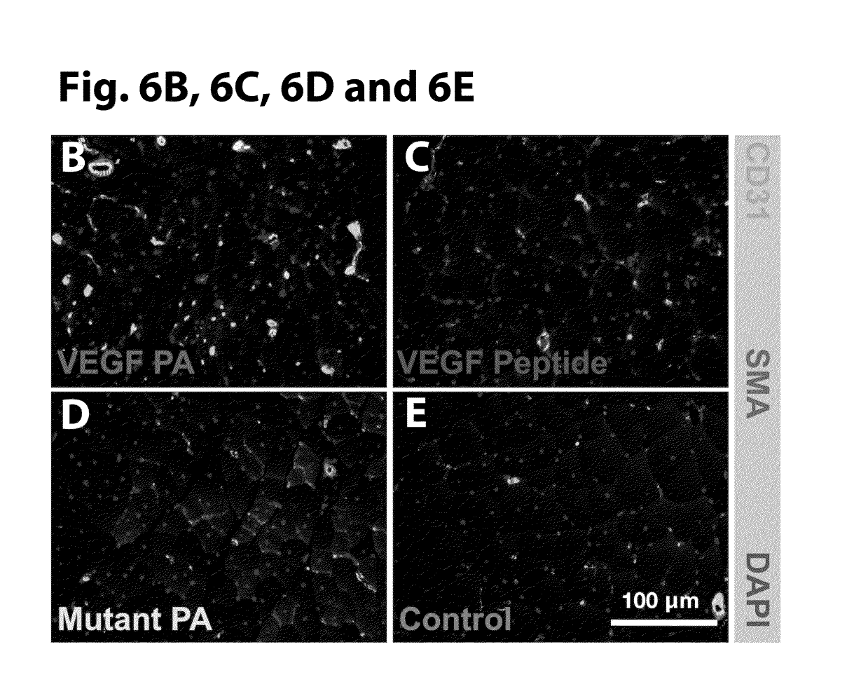

LDPI was performed to assess tissue perfusion in the ischemic hind-limb (FIG. 6A). As shown, treatment with VEGF-PA significantly enhances the recovery of tissue perfusion following treatment. At 14 days after induction of ischemia, animals treated with VEGF-PA had a perfusion ratio (0.76±0.05) that was significantly greater than that for animals treated with the VEGF peptide (0.54±0.06, P<0.01), mutant PA (0.42±0.05, P<0.01) or a control injection of saline (0.53±0.08, P<0.05). At day 28, animals treated with VEGF-PA continued to have a significantly higher perfusion ratio (0.72±0.05) than animals treated with the VEGF peptide (0.52±0.06, P<0.05), mutant PA (0.48±0.05, P<0.05), and saline (0.52±0.05, P<0.05).

In order to determine whether the beneficial effects of VEGF-PA treatment on recovery of blood flow, motor function and tissue salvage were associated with an effect on the microcirculation of the ischemic limb muscle, the number of CD31+ capillaries in the ischemic limb muscle was quantified using immunohistological staining of the tissue harvested at day 28 (FIG. 6B-6E). There was a significant increase in the number of CD31+ capillaries in animals treated with VEGF-PA (1582±76 /mm2) compared to treatment with VEGF peptide (949±48/mm2, P<0.001), mutant PA (954±81/mm2, P<0.001) and saline (893±53 /mm2, P<0.001). Of note, there was no effect on the number of CD31+/smooth muscle actin+ arterioles, also known as muscularized or mature capillaries. This pro-angiogenic effect of VEGF-PA on the microcirculation is consistent with its angiogenic activity in the CAM assay, and reinforces its therapeutic efficacy for ischemic tissue repair.

Overall, the improvements in tissue perfusion, limb salvage, motor function, and capillarization point to the therapeutic utility of VEGF-PA for ischemic tissue repair. Thus, VEGF-PA nanostructures are identified here as a promising synthetic therapeutic strategy for ischemic cardiovascular disease.

### Discussion

Ischemic cardiovascular diseases remain a growing socioeconomic burden. One emerging therapeutic concept aims to reconstitute the microvasculature in ischemic tissue by use of pro-angiogenic factors, termed therapeutic angiogenesis. This strategy has been applied in the form of protein or gene therapies, with a focus on the delivery of highly potent angiogenic factors. VEGF is one of the most promising candidates for microvascular strategies due to its high angiogenic potency. However, clinical trials to date have not convincingly demonstrated efficacy [4, 5]. One potential reason for the failures of protein-based therapies for ischemic tissue is the inadequate retention of protein in the target zone, with some studies finding protein retention in the tissue to be on the order of minutes to a few hours, depending on delivery route [32-34]. Maintaining adequate levels of the protein in the ischemic tissue to achieve the appropriate temporal signaling would necessitate repeated delivery over a prolonged period of time, making these therapies technically challenging and necessitating cost-prohibitive quantities of protein [4].

The data herein confirm the utility of bioactive and biodegradeable nanostructures as a therapy to promote angiogenesis. The display on the surface of these nanofibers of a peptide mimic of VEGF demonstrates enhanced signaling and bioactivity through activation of VEGF receptors and consequent functional outcomes for endothelial cells in vitro. The pro-angiogenic activity of this system was further substantiated in vivo using the CAM assay. Finally, evaluation of the regenerative capacity of these VEGF-PA nanostructures in a murine hind-limb ischemia model reveals improved tissue perfusion, limb motor function, limb salvage, and capillarization of the ischemic limb. The demonstrated efficacy suggests further consideration of these systems as an alternative therapy to protein- or gene-based strategies currently being evaluated for ischemic cardiovascular diseases.

As mentioned previously, the major issues raised with the clinical application of VEGF or other recombinant proteins are specific target tissue retention, limited production resources, and cost. Bioactive PAs provide a potential means to overcome these obstacles. Even though PAs are biodegradable by design and thus will be eventually broken down into natural products, they do remain in the tissue on the order of weeks following injection [23, 27]. This is a substantial improvement when compared to the retention time of VEGF protein of, at most, a few hours. This prolonged presence and bioavailability in the target zone could circumvent the need for serial protein deliveries and the additional pain and suffering this would entail. Another consideration is production cost which has been speculated to be prohibitively expensive to the clinical implementation of efficacious protein-based therapies [4]. The PA could reduce this, in principle, as serial dosing may not be necessary due to enhanced retention. Moreover, the cost per equivalent bioactive epitope for the dose of PA used in the in vivo studies herein is much less than for the whole protein. This makes matching the bioactive dose of the VEGF-PA using VEGF protein very cost-prohibitive even in small animals. The results we have demonstrated here for bioactivity and therapeutic efficacy of a pro-angiogenic PA designed to signal through VEGF receptors point to the translational potential of this therapeutic strategy.

In sum, the data presented herein confirm that a polyvalent self-assembling nanofiber displaying a known VEGF mimicking sequence is efficacious in a hind-limb ischemia model of cardiovascular disease. The observed functional recovery is likely linked to the pro-angiogenic, VEGF mimetic behavior of the VEGF-PA nanostructures established both in vitro and in vivo. Presentation of the mimetic epitope on the polyvalent nanofiber leads to more efficient and effective VEGF signaling compared to the bioactive peptide alone. Thus, these bioactive nanostructures represent a promising cost-effective synthetic therapeutic strategy to regenerate microcirculation and restore perfusion to ischemic tissue in cardiovascular diseases.

### INDUSTRIAL APPLICABILITY

The VEGF-PA described herein represent a synthetic, cost-effective alternative to traditional protein therapies, and demonstrate a pro-angiogenic capacity in studies thus far. A pro-angiogenic PA find utility in the context of therapy for cardiovascular disease, including ischemic disease from coronary artery and peripheral arterial disease. Such PAs may present therapeutic effects in multiple other applications including, for example, the treatment of (chronic) wounds, enhancing the integration of skin grafts, or serving in a therapeutic capacity where enhancing microcirculation would be advantageous. The mimetic PAs of the present invention may also be mixed with traditionally non-bioactive or minimally bioactive tissue engineering scaffolds to produce hybrid scaffolds with an enhanced potential for rapid vascularization and tissue integration. Additionally, such PAs may be used to enhance vasculature in a transplant bed, increasing the likelihood of survival for transplanted tissues, pre-cultivated cells, or tissue-engineered constructs. Such PAs may also find utility as a coating to assist with the integration and endothelialization of stents, vascular grafts, and medical devices.

### REFERENCES

All publications, patent applications, patents and other references mentioned herein are incorporated by reference in their entirety, including:
1. Rosamond W, Flegal K, Furie K, Go A, Greenlund K, Haase N, et al. Heart disease and stroke statistics-2008 update: a report from the American Heart Association Statistics Committee and Stroke Statistics Subcommittee. Circulation 2008 Jan. 29; 117(4):e25-146.
2. Folkman J. Tumor angiogenesis: therapeutic implications. N Engl J Med 1971 Nov. 18; 285(21):1182-1186.
3. Folkman J. Angiogenesis: an organizing principle for drug discovery? Nat Rev Drug Discov 2007 April; 6(4):273-286.
4. Ferrara N, Gerber H P, LeCouter J. The biology of VEGF and its receptors. Nat Med 2003 June; 9(6):669-676.
5. Gupta R, Tongers J, Losordo D W. Human studies of angiogenic gene therapy. Circ Res 2009 Oct. 9; 105(8):724-736.
6. Yla-Herttuala S, Rissanen T T, Vajanto I, Hartikainen J. Vascular endothelial growth factors: biology and current status of clinical applications in cardiovascular medicine. J Am Coll Cardiol 2007 Mar. 13; 49(10):1015-1026.
7. Tongers J, Roncalli J G, Losordo D W. Therapeutic angiogenesis for critical limb ischemia: microvascular therapies coming of age. Circulation, 2008. p. 9-16.
8. Silva E A, Mooney D J. Effects of VEGF temporal and spatial presentation on angiogenesis. Biomaterials 2009 Nov. 9.
9. Simons M, Ware J A. Therapeutic angiogenesis in cardiovascular disease. Nat Rev Drug Discov 2003 November; 2(11):863-871.
10. Rissanen T T, Yla-Herttuala S. Current status of cardiovascular gene therapy. Mol Ther 2007 July; 15(7):1233-1247.
11. D'Andrea L D, Del Gatto A, Pedone C, Benedetti E. Peptide-based molecules in angiogenesis. Chem Biol Drug Des 2006 February; 67(2):115-126.
12. Ferrara N, Kerbel R S. Angiogenesis as a therapeutic target. Nature 2005 Dec. 15; 438(7070):967-974.
13. D'Andrea L D, Iaccarino G, Fattorusso R, Sorriento D, Carannante C, Capasso D, et al. Targeting angiogenesis: structural characterization and biological properties of a de novo engineered VEGF mimicking peptide. Proc Natl Acad Sci USA 2005 Oct. 4; 102(40):14215-14220.
14. Diana D, Ziaco B, Colombo G, Scarabelli G, Romanelli A, Pedone C, et al. Structural determinants of the unusual helix stability of a de novo engineered vascular endothelial growth factor (VEGF) mimicking peptide. Chemistry 2008; 14(14):4164-4166.
15. Stupp S I. Biomaterials for regenerative medicine. Mrs Bulletin 2005 July; 30(7):546-553.
16. Stupp S I, Donners J J J M, Li L S, Mata A. Expanding frontiers in biomaterials. Mrs Bulletin 2005 November; 30(11):864-873.
17. Hartgerink J D, Beniash E, Stupp S I. Self-assembly and mineralization of peptide-amphiphile nanofibers. Science 2001 Nov. 23; 294(5547):1684-1688.
18. Hartgerink J D, Beniash E, Stupp S I. Peptide-amphiphile nanofibers: a versatile scaffold for the preparation of self-assembling materials. Proc Natl Acad Sci USA 2002 Apr. 16; 99(8):5133-5138.
19. Webber M J, Kessler J A, Stupp S I. Emerging peptide nanomedicine to regenerate tisues and organs. J Intern Med 2010; 267:71-88.
20. Velichko Y S, Stupp S I, de la Cruz M O. Molecular simulation study of peptide amphiphile self-assembly. Journal of Physical Chemistry B 2008 Feb. 28; 112(8):2326-2334.
21. Beniash E, Hartgerink J D, Storrie H, Stendahl J C, Stupp S I. Self-assembling peptide amphiphile nanofiber matrices for cell entrapment. Acta Biomater 2005 July; 1(4):387-397.
22. Muraoka T, Koh C Y, Cui H, Stupp S I. Light-triggered bioactivity in three dimensions. Angew Chem Int Ed Engl 2009; 48(32):5946-5949.

While the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. For instance, various peptide amphiphiles have been described in conjunction with specific amino acid residues; however, other residues can be used herewith to promote a particular tissue growth and regeneration on the nanostructures prepared therefrom. Likewise, while the present invention has been described as applicable to biomedical or tissue engineering use, other advantages and features will become apparent from the claims filed hereafter, with the scope of such claims to be determined by their reasonable equivalents, as would be understood by those skilled in the art. Thus, the invention is intended to be defined not by the above description, but by the following claims and their equivalents.

1. A peptide amphiphile consisting of the following segments: (1) a mimetic peptide segment comprising a VEGF agonist that binds to or interacts with a VEGF receptor; (2) a spacer segment that confers both solubility and flexibility to the peptide; (3) a beta-sheet forming, structural peptide segment, and (4) a non-peptide lipophilic segment.

2. The peptide amphiphile of claim 1, wherein said mimetic peptide segment is selected from KLTWQELYQLKYKGI (SEQ ID NO: 2) or D(IGKYKLQYLEQWTLK) (SEQ ID NO: 28)

3. The peptide amphiphile of claim 1 wherein said spacer segment is selected from peptides of the form (Xaa)m-(Gly)n , wherein m and n are integers that independently range between 0 and 5, wherein Xaa is an amino acid residue selected from those with basic side-chains.

4. The peptide amphiphile of claim 4 wherein the spacer peptide segment is KKKG (SEQ ID NO: 6).

5. The peptide amphiphile of claim 1 wherein said structural peptide segment is selected from peptides of the form (XA)Na(XB)Nb wherein XA and XB are selected from A, L, V and G and Na and Nb are 2, 3 or 4.

6. The peptide amphiphile of Claim I wherein the structural peptide segment is VVAA (SEQ ID NO: 19).

7. The peptide amphiphile of claim 1 wherein the lipophilic segment is comprised of a single, saturated, linear acyl group of the formula: CH3(CH2)n-2C(O)—, wherein n=6□22.

8. The peptide amphiphile of claim 8, wherein n=16.

9. The peptide amphiphile of claim 8, wherein the lipophilic segment is covalently linked to the epsilon amine of a lysine residue.

10. A peptide amphiphile consisting of the following structure (SEQ ID NO: 3):

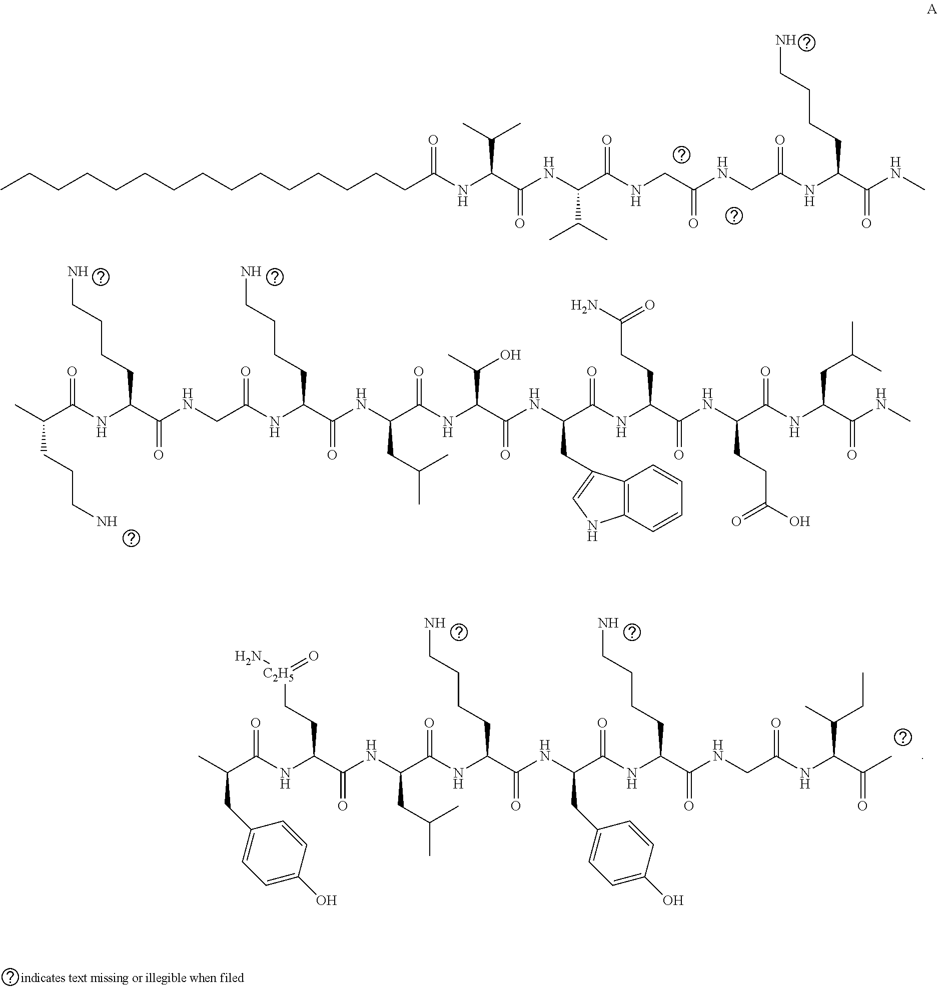

11. A composition comprising one or more of the peptide amphiphiles of claim 1.

12. The composition of claim 12, dispersed in an aqueous medium.

13. A method of treating a patient afflicted with a disease or disorder characterized by poor circulation and blood vessel formation, said method comprising the step of administering a peptide amphiphile of claim 1 or a composition of claim 12 to a target site within the body of said patient in need.

14. The method of claim 14, wherein said disease is peripheral artery disease and said target site is an occluded vessel.

15. The method of claim 14, wherein said disorder is ischemia and said target site is a chronic ischemic wound.

16. A method of treating an ischemic disease or defect in a patient in need thereof, said method comprising the step of administering a composition Claim I to said patient.

17. The method of claim 17 wherein said composition is administered intravenously.

18. The method of claim 17 wherein said composition is administered intramuscularly.

19. The method of claim 17, wherein the patient is human.

20. The method of claim 17, wherein the patient is an animal.

21. The method of claim 17, wherein the patient is a horse, dog, sheep, goat, or cow.

22. A method of regenerating vasculature, said method comprising the step of administering to a patient in need thereof a composition of claim 12.

23. A substrate coated with self-assembled micelles formed by the peptide amphiphile of claim 1.


SEQUENCE LISTING (30 sequences)

SEQ ID NO: 1 - protein, 8 aa, Artificial Sequence
  Description of Artificial Sequence Synthetic peptide
VVAAKKKG

SEQ ID NO: 2 - protein, 15 aa, Artificial Sequence
  Description of Artificial Sequence Synthetic peptide
KLTWQELYQLKYKGI

SEQ ID NO: 3 - protein, 23 aa, Artificial Sequence
  Description of Artificial Sequence Synthetic Peptide
VVAAKKKGKLTWQELYQLKYKGI

SEQ ID NO: 4 - protein, 15 aa, Artificial Sequence
  Description of Artificial Sequence Synthetic Peptide
KLTWQELYQLKYKGI

SEQ ID NO: 5 - protein, 23 aa, Artificial Sequence
  Description of Artifiial Sequence Synthetic peptide
VVAAKKKGKLTAQELVFLKVKGI

SEQ ID NO: 6 - protein, 4 aa, Artificial Sequence
  Description of Artificial Sequence Synthetic peptide
KKKG

SEQ ID NO: 7 - protein, 6 aa, Artificial Sequence
  Description of Artificial Sequence Synthetic peptide
VVVAAA

SEQ ID NO: 8 - protein, 6 aa, Artificial Sequence
  Description of Artificial Sequence Synthetic peptide
AAAVVV

SEQ ID NO: 9 - protein, 6 aa, Artificial Sequence
  Description of Artificial Sequence Synthetic peptide
LLLAAA

SEQ ID NO: 10 - protein, 6 aa, Artificial Sequence
  Description of Artificial Sequence Synthetic peptide
VVVVVV

SEQ ID NO: 11 - protein, 6 aa, Artificial Sequence
  Description for Artificial Sequence Synthetic peptide
VVVLLL

SEQ ID NO: 12 - protein, 6 aa, Artificial Sequence
  Description of Artificial Sequence Synthetic peptide
LLLVVV

SEQ ID NO: 13 - protein, 6 aa, Artificial Sequence
  Description of Artificial Sequence Synthetic peptide
AAAAAA

SEQ ID NO: 14 - protein, 7 aa, Artificial Sequence
  Description of Artificial Sequence Synthetic peptide
AAAAGGG

SEQ ID NO: 15 - protein, 6 aa, Artificial Sequence
  Description of Artificial Sequence Synthetic peptide
LLLLLL

SEQ ID NO: 16 - protein, 6 aa, Artificial Sequence
  Description of Artificial Sequence Synthetic peptide
AAAGGG

SEQ ID NO: 17 - protein, 6 aa, Artificial Sequence
  Description of Artificial Sequence Synthetic peptide
LLLGGG

SEQ ID NO: 18 - protein, 6 aa, Artificial Sequence
  Description of Artificial Sequence Synthetic peptide
AAALLL

SEQ ID NO: 19 - protein, 4 aa, Artificial Sequence
  Description of Artificial Sequence Synthetic peptide
VVAA

SEQ ID NO: 20 - protein, 5 aa, Artificial Sequence
  Description of Artificial Sequence Synthetic peptide
VVVAA

SEQ ID NO: 21 - protein, 5 aa, Artificial Sequence
  Description of Artificial Sequence Synthetic peptide
VVAAA

SEQ ID NO: 22 - protein, 6 aa, Artificial Sequence
  Description of Artificial Sequence Synthetic peptide
VVVVAA

SEQ ID NO: 23 - protein, 4 aa, Artificial Sequence
  Description of Artificial Sequence Synthetic peptide
AAVV

SEQ ID NO: 24 - protein, 5 aa, Artificial Sequence
  Description of Artificial Sequence Synthetic peptide
AAVVV

SEQ ID NO: 25 - protein, 5 aa, Artificial Sequence
  Description of Artificial Sequence Synthetic peptide
AAAVV

SEQ ID NO: 26 - protein, 6 aa, Artificial Sequence
  Description of Artificial Sequence Synthetic peptide
AAVVVV

SEQ ID NO: 27 - protein, 15 aa, Artificial Sequence
  Description of Artificial Sequence Synthetic peptide
IGKYKLQYLEQWTLK

SEQ ID NO: 28 - protein, 15 aa, Artificial Sequence
  Description of Artificial Sequence Synthetic peptide
IGKYKLQYLEQWTLK

SEQ ID NO: 29 - protein, 15 aa, Artificial Sequence
  Description of Artificial Sequence Synthetic peptide
KLTWEELYELKYKGI

SEQ ID NO: 30 - protein, 23 aa, Artificial Sequence
  Description of Artificial Sequence Synthetic peptide
VVAAKKKGKLTWEELYELKYKGI